SEQUENCE LISTING (12 sequences)

SEQ ID NO: 1 - DNA, 20 bp, Artificial Sequence
  NLRP3 forward primer
gtgtttcgaatcccactgtg

SEQ ID NO: 2 - DNA, 22 bp, Artificial Sequence
  NLRP3 reverse primer
tctgcttctcacgtactttctg

SEQ ID NO: 3 - DNA, 20 bp, Artificial Sequence
  IL1beta forward primer
atgcacctgtacgatcactg

SEQ ID NO: 4 - DNA, 21 bp, Artificial Sequence
  IL1beta reverse primer
acaaaggacatggagaacacc

SEQ ID NO: 5 - DNA, 20 bp, Artificial Sequence
  IL6 forward primer
ccactcacctcttcagaacg

SEQ ID NO: 6 - DNA, 22 bp, Artificial Sequence
  IL6 reverse primer
catctttggaaggttcaggttg

SEQ ID NO: 7 - DNA, 18 bp, Artificial Sequence
  TNFalpha forward primer
actttggagtgatcggcc

SEQ ID NO: 8 - DNA, 20 bp, Artificial Sequence
  TNFalpha reverse primer
gcttgagggtttgctacaac

SEQ ID NO: 9 - DNA, 20 bp, Artificial Sequence
  IL18 forward primer
cattgaccaaggaaatcggc

SEQ ID NO: 10 - DNA, 24 bp, Artificial Sequence
  IL18 reverse primer
cacagagatagttacagccatacc

SEQ ID NO: 11 - DNA, 20 bp, Artificial Sequence
  18S rRNA forward primer
gtaacccgttgaaccccatt

SEQ ID NO: 12 - DNA, 20 bp, Artificial Sequence
  18S rRNA reverse primer
ccatccaatcggtagtagcg

(19) United States
(12) Patent Application Publication
(10) Pub. No.: US 2023/0263817 A1
(43) Pub. Date: Aug. 24, 2023

(54) HIGH DOSE NICOTINAMIDE ADENINE DINUCLEOTIDE (NAD) PRECURSOR REGIMENS FOR REDUCTION OF INFLAMMATION IN HUMAN PATIENTS WITH PREEXISTING INFLAMMATION

(71) Applicant: University of Washington, Seattle, WA (US)

(72) Inventors: Rong Tian, Seattle, WA (US); Dennis Ding-Hwa Wang, Seattle, WA (US); Bo Zhou, Seattle, WA (US); Kevin Douglas O'Brien, Seattle, WA (US)

(73) Assignee: University of Washington, Seattle, WA (US)

(21) Appl. No.: 18/024,032

(22) PCT Filed: Aug. 27, 2021
(86) PCT No.: PCT/US2021/047993

§ 371 Date: Feb. 28, 2023

(51) Int. Cl. A61K 31/706; G01N 33/68; G01N 33/50; A61P 29/00

(52) U.S. Cl. CPC A61K 31/706; G01N 33/6869; G01N 33/5094; A61P 29/00; G01N 2333/5412; G01N 2333/545; G01N 2800/325; G01N 2800/7095

(57) ABSTRACT

The current disclosure provides use of high dose nicotinamide adenine dinucleotide (NAD) precursor regimens for reduction of inflammation in human patients with preexisting inflammation. The NAD precursor can be nicotinamide riboside (NR) and the high dose regimen can include at least 1000 or 2000 mg/day for at least 9 days.

### CROSS-REFERENCE TO RELATED APPLICATION

This application is a US National Phase Application which claims priority to International Patent Application No. PCT/US2021/047993 filed on Aug. 27, 2021, which claims priority to U.S. Provisional Patent Application No. 63/071,778 filed on Aug. 28, 2020, the contents of both of which are incorporated herein by reference in their entirety as if fully set forth herein.

### STATEMENT REGARDING FEDERALLY SPONSORED RESEARCH OR DEVELOPMENT

This invention was made with government support under Grant Nos. HL126209 and HL144937, awarded by the National Institutes of Health. The government has certain rights in the invention.

### REFERENCE TO SEQUENCE LISTING

The Sequence Listing associated with this application is provided in text format in lieu of a paper copy and is hereby incorporated by reference into the specification. The name of the text file containing the Sequence Listing is 2UP2655.txt. The text file is 3 KB, was created on Feb. 28, 2023 and is being submitted electronically via EFS-Web.

### FIELD OF THE DISCLOSURE

The current disclosure provides use of a high dose nicotinamide adenine dinucleotide (NAD) precursor regimen to reduce inflammation in human patients with preexisting inflammation.

### BACKGROUND OF THE DISCLOSURE

According to the World Health Organization (WHO), chronic inflammatory diseases are the greatest threat to human health with 3 out of 5 of all deaths globally being attributable to inflammation-related disease. Chronic inflammatory diseases include stroke, cardiovascular disease, chronic respiratory disease, cancer, obesity, diabetes, autoimmune disease, and allergies.

Inflammation involves activation of the immune system in response to harmful stimuli, such as pathogens, infections, stimulants, or cellular damage. Inflammation-inducing pathogen-associated molecular patterns (PAMPs) and danger-associated molecular patterns (DAMPs) are recognized by pattern recognition receptors (PRRs) which upon activation, transduce signals intracellularly. This leads to the release of pro-inflammatory cytokines including tumor necrosis factor-alpha (TNF-α), interleukin (IL)-1, IL-6 and IL-10, and type 1 interferons (IFNs). Those pro-inflammatory cytokines further bolster inflammation through feedback loops.

Nicotinamide adenine dinucleotide (NAD) is a coenzyme that is critical to cell and organismal function. In addition to serving as a carrier for redox reactions, NAD is an essential cofactor in several non-redox reactions which play important roles in transcription regulation, energy metabolism modulation, cell survival, DNA repair, inflammation, circadian rhythm regulation modulation of chromatin structure transcription, replication, and recombination. Supplementation with the NAD precursor, nicotinamide riboside (NR), has been shown to ameliorate several NAD-related physiological stresses in mice such as brain inflammation in diabetic mice, aging-induced nonalcoholic fatty liver disease (NAFLD)-like hepatic dysfunction in mice, retinal degeneration, inflammation and mitochondrial markers in AML12 mouse hepatocytes, and oxidative stress and organ injury in mouse sepsis models. Although NR has been shown to lower the levels of proinflammatory cytokines in animal models, the clinical safety and/or benefit does not translate directly to humans. For example, human digestion and the gut microbiome affect the distribution of NAD precursors in ways that are not yet fully characterized (Gazzaniga et al. Microbiol Mol Biol Rev. 2009, 73:529-541). The dose of NAD precursor used in animal studies is at least an order of magnitude higher than the highest dose used in human on the per kilogram body weight basis (Tong et al., Circ Res 2021, 128(11):1629-1641 and Zhou et al., J Clin Invest 2020, 130(11):6054-6063). Furthermore, while proinflammatory cytokines such as TNFα, IL1, IL6, and IL18 have been demonstrated to have negative-inotropic effects in various experimental models, results of anti-TNFα clinical trials in heart failure were discouraging because the lowering of TNFα did not create a beneficial therapeutic effect (Chung E S, et al., Circulation. 2003, 107(25):3133-40; Mann D L, et al., Circulation. 2004, 109(13):1594-602).

Accordingly, before the current disclosure, the safety and efficacy of high dose NAD precursor regimens in human patients with preexisting inflammation was not known and could not have been reasonably predicted.

### SUMMARY OF THE DISCLOSURE

The present disclosures provides use of high dose nicotinamide adenine dinucleotide (NAD) precursor regimens for the reduction of mitochondria-mediated inflammation in human patients with preexisting inflammation. In certain examples, the NAD precursor is nicotinamide riboside, the high dose regimen is 2000 mg/day for at least 5 days, and the human patient is a Stage D heart failure patient. Importantly, the safety and utility of such high doses of NR in humans with preexisting inflammation had to be assessed. As disclosed herein, such doses are well-tolerated by humans and serve to reduce inflammation in peripheral blood mononuclear cells (PBMCs), as measured by mitochondrial respiration and proinflammatory marker expression.

It is also pointed out that targeting mitochondria-mediated inflammation utilizing a high dose regimen of NAD precursors is distinct from targeting a specific cytokine for reduction. The targeting of mitochondrial-mediated inflammation has a broader coverage of inflammatory mediators, and furthermore, is unlikely to cause an imbalance in cytokine levels which can occur when targeting only one cytokine.

### BRIEF DESCRIPTION OF THE DRAWINGS

FIG. 1. Baseline characteristics of study subjects of Experimental Example 1 (published as Zhou B, et al., J Clin Invest. 2020, 130:6054-6063). LVEF: left ventricular ejection fraction. CM: cardiomyopathy. HTN: hypertension. DM: diabetes mellitus. Data shown in means±SD.

FIGS. 2A-2D: Heart failure is associated with a reduced PBMC maximal respiration and elevated pro-inflammatory cytokine gene expression. (2A) Representative oxygen consumption rate (OCR) plot upon various inhibitor injections in a standard Seahorse Mito Stress Test, comparing PBMCs from healthy (N=19) and Stage D HF (N=19) subjects. Oligomycin A: inhibitor of complex V. FCCP (Carbonyl cyanide-4-(trifluoromethoxy)phenylhydrazone): uncoupling agent by permeabilizing inner mitochondrial membrane. Antimycin A: inhibitor of complex Ill. Rotenone: inhibitor of complex I. (2B) Basal and (2C) FCCP-induced maximal respiration of PBMCs from healthy and Stage D HF subjects, respectively. (2D) Relative mRNA levels of NLRP3 and pro-inflammatory cytokines of PBMC of healthy and Stage D HF subjects by RT-qPCR. NLRP3 (Healthy N=9, HF N=9), IL1p (N=9, N=9), IL6 (N=12, N=11), IL18 (N=7, N=9), TNFα (N=9, N=9). Mean mRNA level of healthy subjects normalized to 1. mRNA data analyzed with unpaired nonparametric t test. All data shown in means+/−SEM.

FIG. 3. Monocyte and lymphocyte counts of the 19 Stage D HF subjects by complete blood count (CBC). For 18 out of 19 subjects, the CBC was done within 15 days (mean 7.7 days) prior to the experimental blood sampling. For the remaining subject, the CBC was done 30 days prior to experimental blood sampling.

FIGS. 4A-4C. (4A) Maximal respiration of healthy PBMCs post 4-hour vehicle or 10 ng/mL LPS treatment with or without 1 mM NR. N=4. Data analyzed with 1-way ANOVA with multiple comparisons. (4B) Relative mRNA levels of NLRP3 and pro-inflammatory cytokines of healthy PBMCs post 4-hour vehicle or 10 ng/mL LPS treatment. P-value determined by paired two-tailed t test. (4C) Percent change of NLRP3 and cytokine mRNA level of healthy PBMCs post 4-hour treatment of 10 ng/mL LPS with 1 mM NR treatment relative to LPS alone. *P-value cut off of 0.05 by paired two-tailed t-test. ns: not significant. All data shown in means +/−SEM.

FIGS. 5A-5F. Mitochondrial DAMP induces PBMC respiratory impairment and inflammatory cytokine gene expression, and the latter can be partially attenuated by inhibition of the NLRP3 inflammasome axis. (5A) Schematics of the MitoDAMP purified from human myocardial tissue used in the in vitro experiments. (5B) Relative mRNA levels of NLRP3 and pro-inflammatory cytokines of healthy PBMCs post 4-hour treatments of vehicle or MitoDAMP. Vehicle normalized to 1. P-value was determined by paired 2-tailed t-test. (5C) Mitochondrial ROS levels of healthy PBMC post MitoDAMP treatment. 0 hour normalized to 100%. P-value cut off of 0.05 was determined by paired 2-tailed t-test. (5D) Maximal respiration of healthy PBMC post 4-hour treatment of vehicle or MitoDAMP with or without 0.5 mM MitoTempo, N=4. P-value determined by one-way ANOVA with multiple pairwise comparisons. (5E) Percent change of NLRP3 and cytokine mRNA levels of healthy PBMCs post 4-hour treatment of MitoDAMP with 0.5 mM MitoTempo (N=4) or 1 uM MCC950 (N=5) relative to MitoDAMP alone. P-value was determined by paired 2-tailed t-test. (5F) Maximal respiration of healthy PBMC post 4-hour treatments of vehicle or MitoDAMP with or without 1 μM MCC950. N=3. P-value determined by one-way ANOVA with multiple pairwise comparisons. ns: not significant. All data shown in means+/−SEM.

FIGS. 6A-6F. Secreted IL6 from MitoDAMP stimulation impairs mitochondrial respiration by reducing Complex I activity. (6A) Secreted IL6 protein level by ELISA of healthy PBMC post 2-hour (N=3) or 4-hour (N=6) treatment of vehicle or MitoDAMP. P-value determined by paired 2-tailed t-test. (6B) Maximal respiration of healthy PBMC post 4-hour treatments of vehicle or MitoDAMP with or without 100 μM LMT28, a specific inhibitor of the IL6 receptor b (GP 130). N=4. (4C) Maximal respiration of healthy PBMC post 4-hour treatments of vehicle or increasing concentrations of human recombinant IL6. N=4. (6B) and (6C) were analyzed with 1-way ANOVA with multiple pairwise comparisons. (6D) Representative Seahorse plot of baseline and post-drug treatment OCR of healthy PBMC post 4-hour treatments of vehicle or IL6 (1 ng/mL). FCCP; uncoupling agent by permeabilizing inner mitochondrial membrane; Rotenone (Rot): Complex I inhibitor; Antimycin A (AA): Complex III inhibitor; TMPD/Ascorbate: exogenous electron donor for Complex IV. N=3. (6E) Quantitation of panel D. (6F) Complex I activity of healthy PBMC post 4-hour treatments of vehicle or IL6 (1 ng/mL). N=5. Vehicle normalized to 1. 6E and 6F are analyzed by paired 2-tailed t-test. All data shown in means+/−SEM.

FIG. 7. Maximal respiration of healthy PBMCs post 4-hour treatments of vehicle or increasing concentrations of recombinant human IL1B or IL18. N=4. Data analyzed with 1-way ANOVA with multiple pairwise comparisons against vehicle.

FIG. 8. Maximal Respiration of isolated monocytes post 4-hour treatment of vehicle of 1 ng/mL of recombinant human IL6. N=7. P-value determined by paired 2-tailed t-test.

FIGS. 9A-9C. Nicotinamide riboside (NR) attenuates MitoDAMP-induced PBMC respiratory impairment and pro-inflammatory cytokine production in vitro. (9A) Secreted IL6 protein by ELISA of healthy PBMC post 4-hour treatment of vehicle 1 mM NR in the presence of MitoDAMP. N=4. P-value determined by paired 2-tailed t test. (9B) Percent change of NLRP3 and cytokines mRNA levels of healthy PBMCs post 4-hour treatments MitoDAMP with 1 mM NR relative to MtioDAMP only. P-value was determined by paired 2-tailed t-test. N=4. (9C) Maximal respiration of healthy PMBC post 4 hour treatment of vehicle, MitoDAMP, or MitoDAMP with 1 mM NR. N=4. P-value determined by ordinary 1-way ANOVA with multiple pairwise comparisons. 9B and 9C shown in means+/−SEM.

FIG. 10. Model for DAMP-induced monocyte activation. Without being bound by theory, the “priming” signal involves interaction of MitoDAMP with TLRs to enhance the expression of inflammasome components and pro-inflammatory cytokines via activation of transcription factor NFκB. The secreted IL6 in the priming step feeds back in an autocrine manner to impair mitochondrial respiration by inhibiting Complex I activity and induce mitochondrial ROS production, which leads to the assembly of the NLRP3 inflammasome to active caspase I. Caspase I, in turn, cleaves pro-IL1 to IL1. Secreted IL1 can feedback to further potentiate the NF-κB axis.

FIG. 11. Ratio of mRNA levels of PBMC from Stage D HF participants post 4-hour treatments of vehicle vs 1 mM NR treatments. N=8. P-value determined by paired two-tailed t-test.

FIGS. 12A-12F. NR enhances mitochondrial respiration and reduces proinflammatory cytokine production in human heart failure. (12A) Ratio of basal or maximal respiration of healthy PBMC post 4-hour vehicle vs 1 mM NR treatments. (12B) Ratio of basal or maximal respiration of HF PBMC post 4-hour vehicle vs 1 mM NR treatments. (12C) Whole blood NAD level of Stage D HF subjects pre- or post-5-9 days of oral NR administration. (12D) Basal and (12E) maximal respiration of PBMC of Stage D HF subjects pre- and post-oral NR administration, respectively. (12F) Relative mRNA levels of NLRP3 and cytokines of PBMC of Stage D HF subjects pre- and post-NR administration. Post-NR mRNA level normalized to 1. For (12A) and (12B), P-values were determined by unpaired two-tailed t-test, and data shown in means+/−SEM. For 12C-12F, the P-values were determined by paired two-tailed t-test.

FIG. 13. Background information of Stage D heart failure subjects who underwent NR administration. M: male. CM: cardiomyopathy. LVEF: left ventricular ejection fraction. NR: Nicotinamide Riboside. HTN: hypertension. DM: diabetes mellitus. NICM: Non-ischemic cardiomyopathy.

FIG. 14. Study Participant Flow (CONSORT Diagram. In brief, 952 patients were assessed for eligibility, with 30 participants meeting all Study inclusion/exclusion criteria at Study Visit 1 (Randomization Visit). The participants were randomized to either receive the allocated intervention (30 participants) or not to receive the allocated intervention (3 participants). At the time of follow up, 2 participants discontinued the intervention because of gastrointestinal intolerance and from refusing clinical follow up. The results from 30 patients were analyzed.

FIG. 15. Schedule of Study Procedures. At the initial screening visit, 30 participants were split into the NR group (n=20) or the Placebo group (n=10). Both groups were started with the initial dose of 250 mg orally twice daily, then up-titrated by 250 mg twice daily each week to a final dose of 1000 mg twice daily by Week 3. Participants were continued on the 1000 mg twice daily dose until the final clinic visit on Week 12. The procedures performed at each week as indicated in the table. An X indicates that the procedure was performed in the indicated week.

FIGS. 16A, 16B. (FIG. 16A) Baseline Demographics, Physical Exam and Medical History by Randomized Group. (FIG. 16B) Baseline Demographic and Laboratory Values by Randomized Group.

FIG. 17. Adverse Events (Total and Per-participant) by Randomized Group

FIG. 18. Changes at Week 12 vs. Baseline in Key Laboratory and Clinical Variables, by Randomized Group.

FIGS. 19A-19D. Individual Participant, On-trial NR and NAD+ Levels. Shown are whole blood levels of NR (FIG. 19A) and NAD+(FIG. 19B) for participants randomized to NR (N=19) and placebo (N=10). Data analyzed by paired non-parametric T-test (Wilcoxon signed-rank test). Per-participant, whole blood levels throughout the Study are shown for NR (FIG. 19C) and for NAD+(FIG. 19D). ns: not significant.

FIGS. 20A, 20B. Pharmacokinetics of NR and NAD+ at Week 4. Shown are whole blood values for NR (FIG. 20A) and NAD+(FIG. 20B) for participants randomized to NR or to placebo participants, drawn at the Week 4 visit. Patients had been on the maximum dose of 1000 mg twice daily for 1 week prior to this visit. Baseline (trough) and 4 h post-1000 mg dose (peak) values are shown. While NR levels rose significantly in the NR group over the 4 h post-dose period, the already-elevated NAD+ remained so by 4 h post-dose. This latter finding suggests that NAD+ levels were maximized after at least 1 week on the 1000 mg twice daily dose, and also that the elevation in NAD+ levels persisted for at least 12 h following the previous NR dose. Data analyzed by paired non-parametric T-test (Wilcoxon signed-rank test). ns: not significant.

FIGS. 21A-21D. Exploratory Endpoints. Shown are changes for the NR and placebo groups in functional capacity (six-minute walk distance, 6MWD, FIG. 21A), quality of life (by Minnesota living with heart failure questionnaire score, MLHFQ, FIG. 21B), left ventricular systolic function (by 2-dimensional ejection fraction, 2D-EF, FIG. 21C) and left ventricular diastolic function (by E/e′ ratio, FIG. 21D). The study was not powered to detect differences in these endpoints. There was no statistical significance, between-group changes in these surrogate endpoints (by non-paired t-test).

FIGS. 22A-22C. Effect of NR vs. placebo on basal and maximally-stimulated mitochondrial respiration in PBMCs, and correlation of maximal respiration with increase in whole blood NAD+ levels. Shown are ratios of post-treatment/pre-treatment values for basal (FIG. 22A) and maximal (FIG. 22B) PBMC mitochondrial respiration for the placebo and NR group. No significant, between-group differences were seen in changes in basal and maximal mitochondrial respiration. However, there was wide variability observed in NAD+ response to NR, and, within the NR group there was a very strong correlation between post-/pre-NAD+ levels and post-/pre-maximal mitochondrial respiration (p=0.0002, FIG. 22C). Data analyzed by linear regression model with P value and R² are shown. PBMC: peripheral blood mononuclear cells. FCCP: Carbonyl cyanide-p-trifluoromethoxyphenylhydrazone. OCR: oxygen consumption rate. ns: not significant.

### DETAILED DESCRIPTION

According to the World Health Organization (WHO), chronic inflammatory diseases are the greatest threat to human health with 3 out of 5 of all deaths globally being attributable to inflammation-related disease. Chronic inflammatory diseases include stroke, cardiovascular disease, chronic respiratory disease, cancer, obesity, diabetes, autoimmune disease, and allergies (Pahwa, A. G., et al., StatPearls. 2021).

Inflammation involves activation of the immune system in response to harmful stimuli, such as pathogens, infections, stimulants, or cellular damage. Inflammation-inducing pathogen-associated molecular patterns (PAMPs) and danger-associated molecular patterns (DAMPs) are recognized by pattern recognition receptors (PRRs) (Frank, M. G., et al., Brain Behav. Immun. 2016, 51:99-1018; Zong M., et al., Ann. Rheum. Dis. 2013, 72:1390-1399; Gong T., et al., Nat. Rev. Immunol. 2019:1-18) which upon activation, transduce signals intracellularly. This leads to the release of pro-inflammatory cytokines including tumor necrosis factor-alpha (TNF-α), interleukin (IL)-1, IL-6 and IL-10, and type 1 interferons (IFNs) (Kawai T. and S. Akira, Cell Death Differ. 2006, 13:816-825). Those pro-inflammatory cytokines further bolster inflammation through feedback loops.

Nicotinamide adenine dinucleotide (NAD) is a coenzyme that is critical to cell and organismal function. In addition to serving as a carrier for redox reactions, NAD is an essential cofactor in several non-redox reactions by providing ADP-ribose to catalyze the enzymatic function of two key protein families—the sirtuins and the poly(ADP-ribose) polymerases (PARPs). Sirtuins are deacetylases with important roles in transcription regulation, energy metabolism modulation, cell survival, DNA repair, inflammation, and circadian rhythm regulation (Haigis and Sinclair. Annu Rev Pathol. 2010, 5:253-295; Chang and Guarente. Trends Endocrinol Metab. 2014, 25:138-145). PARPs are involved in DNA repair, modulation of chromatin structurem transcription, replication, and recombination (Morales et al., Crit Rev Eukaryot Gene Expr. 2014, 24(1):15-28).

Supplementation with the NAD precursor, nicotinamide riboside (NR), has been shown to ameliorate several NAD-related physiological stresses in mice such as brain inflammation in diabetic mice, aging-induced nonalcoholic fatty liver disease (NAFLD)-like hepatic dysfunction in mice, retinal degeneration, inflammation and mitochondrial markers in AML12 mouse hepatocytes, and oxidative stress and organ injury in mouse sepsis models. Although NR has been shown to lower the levels of proinflammatory cytokines in animal models, the safety and/or efficacy in humans has not been established and could not reasonably be predicted. For example, human digestion and the gut microbiome affect the distribution of NAD precursors in ways that are not yet fully characterized (Gazzaniga et al. Microbiol Mol Biol Rev. 2009, 73:529-541). The dose of NAD precursor used in animal studies is at least an order of magnitude higher than the highest dose used in human on the per kilogram body weight basis (Tong et al Circ Res 2021 and Zou et al. J Clin Invest 2020). Furthermore, while proinflammatory cytokines such as TNFα, IL1, IL6, and IL18 have been demonstrated to have a negative-inotropic effects in various experimental models, results of anti-TNFα clinical trials in heart failure were discouraging because lowering TNFα levels did not produce a significant clinical benefit. (Chung E S, et al., Circulation. 2003, 107(25):3133-40; Mann D L, et al., Circulation. 2004, 109(13):1594-602).

Accordingly, before the current disclosure, the safety and efficacy of high dose NAD precursor regimens in human patients with preexisting inflammation was not known and could not have been reasonably predicted.

The present disclosures provides use of a high dose regimen of nicotinamide adenine dinucleotide (NAD) precursors for reduction of mitochondria-mediated inflammation in peripheral blood mononuclear cells (PBMC) in human patients with preexisting inflammation. In certain examples, the NAD precursor is nicotinamide riboside, the high dose regimen is 2000 mg/day for at least 5 days, and the human patient has American College of Cardiology/American Heart Association Stage D heart failure (defined as a patient with refractory heart failure requiring advanced intervention (i.e. biventricular pacemakers, left ventricular assist device, transplantation)). Importantly, the safety and utility of such high dose regimens of NR in humans with preexisting inflammation had to be assessed. As disclosed herein, such high dose regimens are well-tolerated by humans and serve to reduce inflammation in PBMCs, as measured by mitochondrial respiration and proinflammatory marker expression. This safety profile is in contrast to the common NAD precursor, vitamin B (niacin) which is not tolerated at 2000 mg/day without significant toxicities. Vitamin B is expressly excluded from the meaning of NAD precursors within the current disclosure.

It is noted that targeting mitochondria-mediated inflammation is distinct from targeting a specific cytokine. It has a broader coverage of inflammatory mediators, and furthermore, it is unlikely to cause an imbalance of cytokine levels which could occur when targeting only one cytokine.

In certain examples, a high dose of a NAD precursor is at least 2 or 3 times its maximum recommended dose when taken as a nutritional supplement. In certain examples, a high dose of a NAD precursor is at least 4 times its maximum recommended dose when taken as a nutritional supplement. In other examples, a high dose of a NAD precursor is at least 3 or 4 times its minimum recommended dose when taken as a nutritional supplement. In other examples, a high dose of a NAD precursor is at least 10 times its minimum recommended dose when taken as a nutritional supplement. Exemplary high doses include at least 750 mg/day, at least 1000 mg/day, at least 1200 mg/day, at least 1500 mg/day, at least 1700 mg/day or at least 2000 mg/day. As indicated previously, in certain examples, a high does regimen includes a high dose of 2000 mg/day, administered for at least 5 days. In other examples, a high dose regimen includes a high dose of 1000 mg/day administered twice daily for at least 5 days. In certain situations, daily doses of 250 mg/day, 500 mg/day, or 750 mg/day can also be used.

The high dose of the NAD precursor within a high dose regimen can be given in one administration per day or can be broken into sub-doses within a day (e.g., 2, 3, 4, or 5 sub-doses). In certain examples, the high doses can be administered daily, every other day, every third day, every fourth day, every fifth day, every sixth day, or weekly as part of a high dose regimen.

In certain examples, the administration of high dose NAD precursors to human subjects is oral administration. In certain examples, the high dose regimen includes administration of the NAD precursor orally at 2000 mg/day NR to human subjects with preexisting inflammation for at least 5 days, at least 12 days, at least two weeks, at least 3 weeks, at least 4 weeks, at least 5 weeks, at least 6 weeks, at least 7 weeks, at least 8 weeks, at least 9 weeks, at least 10 weeks, at least 11 weeks, or at least 12 weeks.

In certain examples, administration of the high dose regimen of a NAD precursor results in an increase in PBMC oxygen consumption rates and a decrease in PBMC NLRP3, IL6, IL1β, IL18, and/or TNFα expression.

Aspects of the disclosure are now described in additional detail as follows: (i) Human Subjects with Preexisting Inflammation, (ii) NAD Precursors, (iii) Compositions for Administration, (iv) Methods of Use, (v) Exemplary Embodiments, (vi) Experimental Examples, and (vii) Closing Paragraphs. These headings are provided for organizational purposes only and do not limit the scope or interpretation of the disclosure.

(i) Human Subjects with Preexisting Inflammation. Part of the current disclosure includes identifying subjects with preexisting inflammation, for example, preexisting inflammation caused by peripheral blood mononuclear cells (PMBCs).

Inflammation refers to one aspect of innate immunity, which is compared to acquired immunity that is specific for each pathogen. Inflammation can be classified as either acute or chronic. Generally speaking, acute inflammation is mediated by granulocytes, and chronic inflammation is mediated by PBMCs, such as monocytes and lymphocytes.

Acute inflammation is the body's first protective response that removes damaging stimuli by maintaining tissue integrity and contributing to tissue repair. It is part of the body's natural defense system against injury and disease, and without acute inflammation, wounds and infections would not heal and progressive destruction of the tissue would jeopardize the organism's survival. Acute inflammatory reactions require constant stimulation to be maintained and must be actively terminated when no longer needed. Thus, acute inflammation abates once the damaging stimulus is removed.

Chronic inflammation persists over a period of time as a result of a persistent inflammatory stimulus in which the host usually fails to completely eliminate the causative agent. Chronic inflammation, however, can also occur in the absence of an appropriate inflammatory stimulus, for example, in the context of autoimmune disease.

Chronic inflammation can be characterized as the simultaneous destruction and healing of tissue from the inflammatory process, the net result of which promotes damage rather than repair. Thus, chronic inflammation is considered a disease state. Since inflammatory responses can occur anywhere in the body, chronic inflammation is associated with the pathophysiology of a wide range of seemingly unrelated disorders that underlie a wide variety of human diseases. Chronic inflammation has been implicated in cardiovascular disease, cancer, allergy, obesity, diabetes, digestive system diseases, degenerative diseases, autoimmune disorders, and Alzheimer's disease.

As indicated, part of the current disclosure includes identifying a human subject who has inflammation. In particular embodiments, the inflammation is caused by PBMCs.

Inflammation caused by PBMCs can be detected by examining a human subject's expression levels of NLRP3, IL6, IL1β, IL18, and/or TNFα.

In certain examples, the identified human subject has preexisting inflammation. Identification of preexisting inflammation can be based on assessing a preexisting inflammation marker profile. The preexisting inflammation marker profile can assess whether the subject has increased NLRP3, IL6, IL1β, IL18, and/or TNFα expression as compared to a “healthy” or “normal” baseline. Healthy or normal baseline levels can be derived from reference populations that do not have inflammation or a condition associated with chronic inflammation. Alternatively, a human subject can be identified as having preexisting inflammation through comparison to a reference level of subjects previously identified as having a chronic PBMC inflammatory state, wherein the subject's test results do not significantly differ from the reference level within a 95% confidence interval.

In particular embodiments, a “baseline” or “reference level” can refer to a standardized value for NLRP3, IL6, IL1β, IL18, and/or TNFα expression which represents a level not associated with any inflammation (baseline) or a level associated with a particular type of inflammation (reference level).

As is understood by one of ordinary skill in the art, determining a baseline cytokine level for healthy patients can vary based on the assay used. Standard baseline (or reference) levels can vary from source to source or from laboratory to laboratory. In particular embodiments, healthy patients have a baseline NLRP3 level. In particular embodiments, healthy patients have an IL6 range of 0-6 pg/mL using Beckman Dxl according to the University of Washington. Other assays may have ranges of healthy levels of, for example, 0-8 pg/mL, 0-10 pg/mL, 0-12 pg/mL, or 0-15 pg/mL IL6. In particular embodiments, healthy patients have an IL1β value of less than 1.0 pg/mL using direct enzyme immunoassay according to the Mayo Clinic (Whicher and Evans, 1990, Clinical Chemistry 36: 1269-1281; and Bevilacqua et al., 1986, PNAS 83:4533-4537). Other assays may have ranges of healthy levels of, for example, less than less than 3.0 pg/mL, less than less than 2.0 pg/mL, or less than less than 0.5 pg/mL IL1β. In particular embodiments, healthy patients have an IL18 range of 0-492 pg/mL (Colafrancesco et al., 2012, Int J Inflamm vol. 2012, 6 pages). In certain examples, this range could also be 0-500 pg/mL, 0-550 pg/mL, or 0-650 pg/mL. In particular embodiments, healthy patients have a TNFα value less than 5.6 pg/mL using electrochemiluminescence via sandwich immunoassay according to the Mayo Clinic (Chunk, 2001, Prog Respir Res 31:242-246; Bienvenu et al., 1998, Toxicology 129:55-61; and Debad et al., 2004, Clinical and Biological Application of ECL in Electrogenerated Chemiluminescence, ed A. J. Bard. Marcel Dekker, New York, pp. 43-78). In particular embodiments, healthy patients have a TNFα level less than 1 pg/mL (Feldman et al., 2000, J Am Coll Cardiol 35(3):537-544). Other assays could have healthy ranges based on, for example, a measure of less than 12 pg/m, less than 10 pg/m, less than 8 pg/m, less than 6 pg/m, or less than 4 pg/m TNFα.

A “dataset” as used herein is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Datasets can be used by an interpretation function to derive a PBMC inflammation score, which can provide a quantitative measure of preexisting inflammation when compared to a baseline or reference level.

Preexisting inflammation refers to the outcome of a first assessment of inflammation in a subject and the diagnosis of preexisting inflammation is made against a non-personal baseline (healthy) or reference level (inflammation). In certain examples, human subjects with preexisting inflammation have an elevated proinflammatory marker profile. An elevated proinflammatory marker profile can include elevated expression of NLRP3 and/or at least one proinflammatory cytokine selected from IL6, IL1β, IL18, and/or TNFα. In certain embodiments, an elevated proinflammatory marker profile can include elevated expression of NLRP3. In certain embodiments, an elevated proinflammatory marker profile can include elevated expression of the proinflammatory cytokines IL6, IL1β, IL18, and TNFα. In certain embodiments, an elevated proinflammatory marker profile can include elevated expression of NLRP3 and elevated expression of the proinflammatory cytokines IL6, IL1β, IL18, and TNFα. In certain examples, elevated means above a healthy threshold within a particular assay.

In particular embodiments, human subjects with preexisting inflammation can have a reference NLRP3 level. In particular embodiments, human subjects with preexisting inflammation can have an IL6 level above 6 pg/mL. In particular embodiments, human subjects with preexisting inflammation can have an IL1p level above 1.0 pg/mL. In particular embodiments, human subjects with preexisting inflammation can have an IL18 level above 492 pg/mL. In particular embodiments, human subjects with preexisting inflammation can have a TNFα level above 5.6 pg/mL. In particular embodiments, human subjects with preexisting inflammation can have a TNFα level at or above 2 pg/mL (Feldman et al., 2000, J Am Coll Cardiol 35(3):537-544).

Persistent inflammation refers to non-resolved inflammation in a subject at a subsequent assessment that is after the first assessment. A finding of PBMC inflammation at the subsequent assessment can be in comparison to a non-personal baseline or reference level or can be in comparison to the human subject's first assessment.

In certain examples, human subjects with preexisting inflammation can have a high inflammation level. High inflammation levels are characterized by a substantially elevated proinflammatory marker profile. A substantially elevated proinflammatory marker profile can include elevated expression of NLRP3 and/or at least one proinflammatory cytokine selected from IL6, IL1β, IL18, and/or TNFα that is at least 2-standard deviations above a baseline level, according to a relevant statistical test. In certain embodiments, a substantially elevated proinflammatory marker profile can include substantially elevated expression of NLRP3. In certain embodiments, a substantially elevated proinflammatory marker profile can include a substantially elevated expression of the proinflammatory cytokines IL6, IL1β, IL18, and TNFα.

In certain embodiments, a substantially elevated proinflammatory marker profile can include substantially elevated expression of NLRP3 and substantially elevated expression of the proinflammatory cytokines IL6, IL1β, IL18, and TNFα.

As indicated, expression levels of at least one of NLRP3, IL6, IL1β, IL18, and/or TNFα can be assessed. These molecules can be measured according to any method known in the art, such as enzyme-linked immunosorbent assay (ELISA), direct enzyme immunoassay, electrochemiluminescence via sandwich assay, western blot analysis, dot blot, northern blot analysis, nuclease protection assay, in situ hybridization, or polymerase chain reaction (PCR).

When assessing a subject for an inflammatory marker profile, it can also be useful to assess the subject's PBMC's oxygen consumption rate. This measure can be useful as a reference level for subsequent measures in the same subject, for example, to assess whether a high dose treatment regimen was administered at a therapeutically effective amount.

“Oxygen consumption rate” or “OCR” refers to the rate of respiration of cells or isolated mitochondria. OCR is generally measured as the amount of oxygen (pmol) consumed per minute (reported as a positive integer). The measurement is generally also normalized by number of cells or total amount of cellular protein.

In some instances, the respiratory capacity is measured by measuring oxygen consumption rate of the isolated cells. For example, the oxygen consumption rate may be measured by determining maximal oxygen consumption rate (Max OCR). In some embodiments, the oxygen consumption rate is measured by determining spare respiratory capacity (SRC) or respiratory control ratio (RCR). In certain embodiments, SRC is determined for PBMCs as the difference between Max OCR and the Basal OCR. In certain embodiments, RCR is determined for isolated mitochondria as calculated as the ratio of state 3 respiration to state 4o respiration (state 3 respiration/state 4o respiration ratio).

In some instances, measuring the respiratory capacity of the isolated circulating blood cells in any of the above-described methods may include (i) measuring a first rate of oxygen disappearance from the medium to determine the Basal OCR; (ii) adding an ATP synthase inhibitor to the medium; (iii) adding a mitochondrial uncoupler to the medium; (iv) measuring a second rate of oxygen disappearance from the medium to determine the Max OCR; and (v) calculating the SRC as the difference between the Max OCR and the Basal OCR. In some instances, measuring respiratory capacity of the isolated circulating blood cells may further include adding a mitochondrial Complex I inhibitor and a mitochondrial Complex II inhibitor after measuring a second rate of oxygen disappearance from the medium, measuring a third rate of oxygen disappearance from the medium to determine a non-mitochondrial oxygen consumption (Non-Mito OCR), and subtracting the Non-Mito OCR from the Basal OCR and the Max OCR before calculating the SRC. In one example, the mitochondrial uncoupler may be carbonyl cyanide-4-(trifluoromethoxy)phenylhydrazone (FCCP). In another example, the ATP synthase inhibitor may be oligomycin. In another example, the mitochondrial Complex I inhibitor may be rotenone. In another example, the mitochondrial Complex II inhibitor may be antimycin.

In certain examples, the identified human subject has: preexisting inflammation, preexisting high inflammation, persistent inflammation, and/or persistently high inflammation.

Human subjects with heart failure are subjects with preexisting inflammation. Heart failure (HF) as used herein refers to a chronic, progressive condition in which the heart muscle is unable to pump enough blood to meet the subject's needs for blood and oxygen. Heart failure is a clinical disorder characterized by congestion and decreased functional capacity. Heart failure may include the left side, right side, or both sides of the heart. Accordingly, in some embodiments, heart failure may be left-sided heart failure (systolic failure or diastolic failure), right-sided heart failure, congestive heart failure, or any combination thereof.

Heart failure may be classified by the New York Heart Association (NYHA) Functional Classification system. The NYHA Functional Classification system may include a functional capacity, which is a description of how the subject feels during physical activity, and an objective assessment. Functional capacity is ranked from class I to class IV, with increasing limitations of physical activity. Objective assessment is ranked from class A to class D, with increasing severity. Four stages of HF have been identified to classify the evolution and progression of the disease. Stage A and Stage B are considered pre-heart failure where Stage A refers to a patient at high risk for developing HF but does not have a structural disorder of the heart. Stage A patients may have a family history of HF or may have a medical condition including hypertension, diabetes, coronary artery disease, metabolic syndrome, or may have a history of alcohol abuse, rheumatic fever, or drug use. Stage B refers to a patient with a structural disorder of the heart but has not developed symptoms of HF. For example, a Stage B patient could be diagnosed with systolic left ventricular dysfunction but does not have any symptoms of HF. Traditional clinical diagnosis for HF includes Stage C and Stage D. Stage C refers to a patient with underlying structural heart disease that has past or current symptoms of HF the condition. In certain examples, Stage D refers to a patient with final stage of the disease who requires specialized treatment strategies such as mechanical circulatory support, continuous inotropic infusions, cardiac transplantation, heart surgery, or palliative or hospice care.

Other examples of human subjects that can present with preexisting inflammation include those with autoimmune diseases (e.g., lupus or rheumatoid arthritis), diabetes, chronic respiratory disease, stroke, cancer, obesity, diabetes, and allergies.

(ii) NAD Precursors. As used herein, NAD precursors increase levels of NAD within PBMC following administration of a high dose regimen. Vitamin B is excluded due to toxicities associated with its administration at a high dose.

In particular embodiments, the NAD precursor is NR (CAS Number 1341-23-7). The chemical structure of NR includes

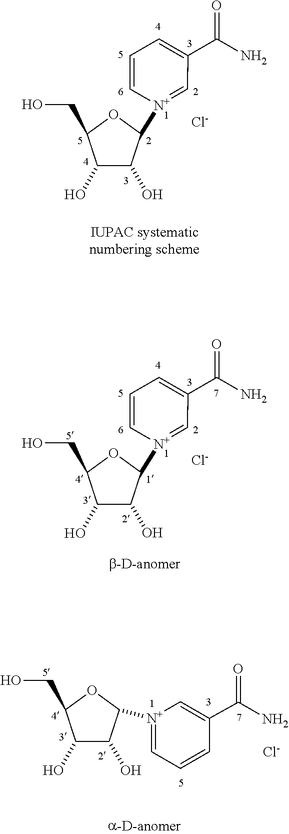 [SMILES: NC(=O)c1ccc[n+]([C@@H]2O[C@H](CO)[C@@H](O)[C@H]2O)c1 ; [Cl-] ; NC(=O)c1ccc[n+]([C@@H]2O[C@H](CO)[C@@H](O)[C@H]2O)c1 ; [Cl-] ; NC(=O)c1ccc[n+]([C@H]2O[C@H](CO)[C@@H](O)[C@H]2O)c1 ; [Cl-]]

NR can be isolated from natural sources or synthesized. Exemplary isolation and synthesis methods are described in Haynes et al., A. J. Chem. Soc. 1957, 3727-3732 and WO 2010/111111. In particular embodiments, NR can be used in its reduced form (NRH) as a 1,4-dihydropyridine compound. Nicotinic acid riboside (NAR) and its reduced form (NARH) can also be used as NAD precursors. For example, US20180362570 describes oxidized and reduced forms of NR with improved stability and bioavailability compared to NR. Particularly described are compounds MP-05, MP-06, MP-07, MP-08, MP-09 and MP-10.

US20180200275 describes that mixing NR with anthocyanin(s) or flavan-3-ol(s) of flavonoids forms positively-charged aggregating molecular forms through co-solvation and improves the oral absorption of NR through the stomach or intestine.

Useful NR derivatives are described in U.S. Ser. No. 10/000,520 and US20060229265.

U.S. Ser. No. 10/000,520 describes a structure

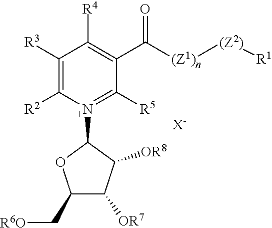

wherein X⁻ is selected from the group including fluoride, chloride, bromide, iodide, formate, acetate, ascorbate, benzoate, carbonate, citrate, carbamate, formate, gluconate, lactate, methyl bromide, methyl sulfate, nitrate, phosphate, diphosphate, succinate, sulfate, trifluoromethanesulfonate, trichloromethanesulfonate, tribromomethanesulfonate, and trifluoroacetate;
Z¹ and Z² are independently NH or oxygen;
n is 0 or 1;

R¹ is selected from the group including hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin 1 ester, vitamin B2 ester, vitamin B6 ester, and —C**H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NRCC(O)R^{C}, —NR^{C}C(O)O(C₁-C₆)alkyl, —NRCC(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C}; R^{A} is selected from the group including —H, —(C₁-C₆)alkyl, —(CH₂)₃—NH—C(NH₂)(═NH), —CH₂C(═O)NH₂, —CH₂COOH, —CH₂SH, —(CH₂)₂C(═O)—NH₂, —(CH₂)₂COOH, —CH₂-(2-imidazolyl), —CH(CH₃)—CH₂—CH₃, —CH₂CH(CH₃)₂, —(CH₂)₄—NH₂, —(CH₂)₂—S—CH₃, phenyl, —CH₂-phenyl, —CH₂—OH, —CH(OH)—CH₃, —CH₂-(3-indolyl), —CH₂-(4-hydroxyphenyl), —CH(CH₃)₂, and —CH₂—CH₃;

R^{B} is hydrogen or —(C₁-C₈)alkyl;

each R^{C} is independently selected from the group including hydrogen, —(C₁-C₈)alkyl, substituted or unsubstituted pyridyl, substituted or unsubstituted 1,4-dihydropyridyl, a radical of the compound of formula (1), and vitamin B7 ester (biotinyl); wherein the substituted pyridyl and substituted 1,4-dihydropyridyl are substituted with one to five substituents independently selected from the group including —(C₁-C₆)alkyl, —(C₂-C₆)alkenyl, —(C₂-C₆)alkynyl, halogen, —CN, —NO₂, —C(O)R^{B}, —C(O)OR^{B}, —C(O)NR^{B}₂, —C(═NR^{B})NR^{B}₂, —OR^{B}, —OC(O)(C₁-C₆)alkyl, —OC(O)O(C₁-C₆)alkyl, —OC(O)NR^{B}₂, —(C₁-C₆)alkylene-NR^{B}₂, —NR^{B}₂, —NR^{B}C(O)R^{B}, —NR^{B}C(O)O(C₁-C₆)alkyl, —NR^{B}C(O)NR^{B}₂, —NR^{B}SO₂NR^{B}, —SR^{B}, —S(O)R^{B}, —SO₂R^{B}, —OSO₂(C₁-C₆)alkyl, —SO₂NR^{B}₂, —(C₁-C₆)perfluoroalkyl, and —(C₁-C₆)alkylene-OR^{B}; R², R³, R⁴, and R⁵ are each independently selected from the group including hydrogen, —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C2}, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NRCC(O)R^{C}, —NR^{C}C(O)O(C₁-C₆)alkyl, —NRCC(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C}; R⁶ is selected from the group including hydrogen, —C(O)R′, —C(O)OR′, —C(O)NHR′, —P(O)(OY¹)(OY²), —P(O)(OY¹)(NHR″), substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin 1 ester, vitamin B2 ester, vitamin B6 ester, and —C**H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C2}, —C(═NR^{C})NR^{C}₂, —OR^{C}, —OC(O)(C₁-C₆)alkyl, —OC(O)O(C₁-C₆)alkyl, —OC(O)NR^{C}₂, (C₁-C₆)alkylene-NR^{C}₂, —N₂, —NR^{C}₂, —NRCC(O)R^{C}, —NR^{C}C(O)O(C₁-C₆)alkyl, —NRCC(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C};

R′ is selected from the group including hydrogen, substituted or unsubstituted —(C₁-C₈)alkyl, substituted or unsubstituted —(C₁-C₈)cycloalkyl, substituted or unsubstituted aryl, substituted or unsubstituted heteroaryl, substituted or unsubstituted heterocycle, vitamin 1 ester, vitamin B2 ester, vitamin B6 ester, choline ester, biotin ester, vitamin A ester, resveratrol ester, aryl(C₁-C₄)alkyl, heterocycle(C₁-C₄)alkyl, and —C**H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NRCC(O)R^{C}, —NR^{C}C(O)O(C₁-C₆)alkyl, —NRCC(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C}; R″ is selected from the group including hydrogen, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle, vitamin 1 ester, vitamin B2 ester, vitamin B6 ester, choline ester, biotin ester, vitamin A ester, resveratrol ester, aryl(C₁-C₄)alkyl, heterocycle(C₁-C₄)alkyl, and —C**H—(R^{A})—CO₂R^{B}; wherein the substituted (C₁-C₈)alkyl, substituted (C₁-C₈)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C}₂, —OR^{C}, —OC(O)(C₁-C₆)alkyl, —OC(O)O(C₁-C₆)alkyl, —OC(O)NR^{C}₂, (C₁-C₆)alkylene-NR^{C}₂, —NR^{C}₂, —NRCC(O)R^{C}, —NR^{C}C(O)O(C₁-C₆)alkyl, —NRCC(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C}; R⁷ and R⁸ are independently selected from the group including hydrogen, —C(O)R′, —C(O)OR′, —C(O)NHR′, substituted or unsubstituted (C₁-C₈)alkyl, substituted or unsubstituted (C₁-C₈)cycloalkyl, substituted or unsubstituted aryl, substituted or unsubstituted heteroaryl, substituted or unsubstituted heterocycle, substituted or unsubstituted aryl(C₁-C₄)alkyl, and substituted or unsubstituted heterocycle(C₁-C₄)alkyl; wherein the substituted (C₁-C₈)alkyl, substituted (C₁-C₈)cycloalkyl, substituted aryl, substituted heteroaryl, substituted heterocycle, substituted aryl(C₁-C₄)alkyl, and substituted heterocycle(C₁-C₄)alkyl are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C}₂, —C(═NR^{C})NR^{C2}, —OR^{C}, —OC(O)(C₁-C₆)alkyl, —OC(O)O(C₁-C₆)alkyl, —OC(O)NR^{C}₂, —(C₁-C₆)alkylene-NR^{C}₂, —NR^{C}₂, —NRCC(O)R^{C}, —NRCC(O)O(C₁-C₆)alkyl, —NRCC(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C}; Y¹ and Y² are independently selected from the group including hydrogen, sodium, potassium, lithium, substituted or unsubstituted (C₁-C₈)alkyl, substituted or unsubstituted (C₁-C₈)cycloalkyl, substituted or unsubstituted aryl, substituted or unsubstituted heteroaryl, and substituted or unsubstituted heterocycle; wherein the substituted (C₁-C₈)alkyl, substituted (C₁-C₈)cycloalkyl, substituted aryl, substituted heteroaryl, and substituted heterocycle are substituted with one to five substituents independently selected from the group including —(C₁-C₆)alkyl, —(C₂-C₆)alkenyl, —(C₂-C₆)alkynyl, halogen, —CN, —NO₂, —C(O)R^{C}, —C(O)OR^{C}, —C(O)NR^{C2}, —C(═NR^{C})NR^{C}₂, —OR^{C}, —OC(O)(C₁-C₆)alkyl, —OC(O)O(C₁-C₆)alkyl, —OC(O)NR^{C}₂, (C₁-C₆)alkylene-NR^{C}₂, —NR^{C}₂, —NRCC(O)R^{C}, —NRCC(O)O(C₁-C₆)alkyl, —NRCC(O)NR^{C}₂, —NR^{C}SO₂NR^{C}, —SR^{C}, —S(O)R^{C}, —SO₂R^{C}, —OSO₂(C₁-C₆)alkyl, —SO₂NR^{C}₂, —(C₁-C₆)perfluoroalkyl, and —(C₁-C₆)alkylene-OR^{C}; or, alternatively, Y¹ and Y² taken together are selected from the group including sodium, potassium, lithium, magnesium, calcium, strontium, and barium; provided that when Z² is NH, the absolute configuration of C** is R or S, or a mixture of R and S;

further provided that when n is 0, Z² is NH, and R¹ is hydrogen, then R⁶, R⁷, and R⁸ are not all simultaneously hydrogen;

further provided that when n is 0, Z² is oxygen, and R¹ is hydrogen, then R⁶, R⁷, and R⁸ are not all simultaneously hydrogen, acetyl, or benzoyl; and further provided that when n is 0, Z² is oxygen, and R¹ is —(C₁-C₈)alkyl, then R⁶, R⁷, and R⁸ are not all simultaneously acetyl or benzoyl.

One example of an NR analogue includes the NR chloride (3-carbamoyl-1-[(2R,3R,4S5R)-3,4-dihydroxy-5-(hydroxymethyl)oxolan-2-yl]-pyrin-1-ylium chloride; also referred to as 1-(β-D-Ribofuranosyl) nicotinamide chloride, which is a salt form of NR; see US20170210774). US20170204131 describes crystalline forms of NR chloride that are chemically stable. These crystalline forms of NR chloride include 3-carbamoyl-1-((2R,3R,4S,5R)-3,4-dihydroxy-5(hydroxymethy)tetrahydrofuran-2-yl)pyridin-1-ium(β-D-NR) chloride crystal, 3-carbamoyl-1-((2R,3R,4S,5R)-3,4-dihydroxy-5(hydroxymethy)tetrahydrofuran-2-yl)pyridin-1-ium(3-D-NR chloride methanolate crystal, and 3-carbamoyl-1-((2S,3R,4S,5R)-3,4-dihydroxy-5(hydroxymethyl)tetra hydrofuran-2-yl)pyridin-1-ium chloride. The first listed is more resistant to decomposition upon heating than other forms.

U.S. Pat. No. 8,106,184 describes the following NR analogues with reduced potential toxicities: O-ethyl NR (OENR), tri-O-acetyl O′-ethyl NR (TAENR), N-dimethyl NR (DMNR), and N-allyl NR (ANR).

Additional NR analogues are described in US20170189433 and U.S. Ser. No. 10/000,519. Examples include:

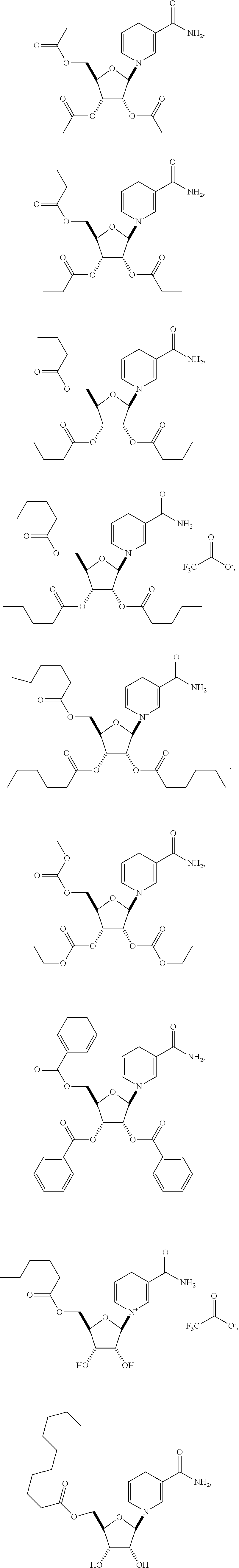

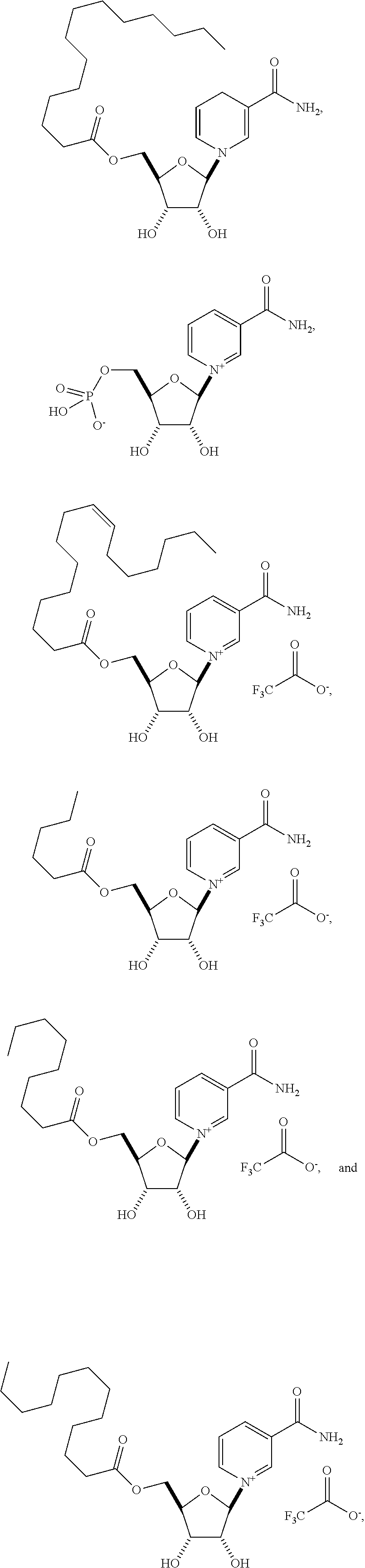

Additional exemplary NR analogues are described in, for example, Bioorg. Med. Chem. Lett. 2002, 12, 1135-1137; Franchetti et al., Bioorg. Med. Chem. Lett. 2004, 14, 4655-4658; and Yang, et al., Med. Chem. 2007, 50, 6458-6461.

In particular embodiments, NAD precursors include any compound including NR (e.g., NR chloride, NR bromide, OENR (O-ethyl nicotinamide riboside), TAENR (tri-O-acetyl O′-ethyl nicotinamide riboside), DMNR (N-dimethyl nicotinamide riboside), and ANR (N-allyl nicotinamide riboside)).

(iii) Compositions for Administration. NAD precursors can be formulated alone or in combination into compositions for administration to subjects. Salts and/or pro-drugs of NAD precursors can also be used.

A pharmaceutically acceptable salt includes any salt that retains the activity of the NAD precursor and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage of a NAD precursor or by hydrolysis of a biologically labile group.

Exemplary generally used pharmaceutically acceptable carriers include any and all absorption delaying agents, antioxidants (e.g., ascorbic acid, methionine, vitamin E), binders, buffering agents, bulking agents or fillers, chelating agents (e.g., EDTA), coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the NAD precursor or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The formulations disclosed herein can be formulated for administration by, for example, oral administration. For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of NAD precursor and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one NAD precursor. Various sustained-release materials have been established and are well known by those of ordinary skill in the art. Sustained-release systems may, depending on their chemical nature, release NAD precursor following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

In particular embodiments, the compositions include NAD precursor of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Compositions disclosed herein can be formulated for administration by, for example, ingestion, injection, infusion, perfusion, or lavage. The compositions disclosed herein can further be formulated for oral, intravenous, intradermal, intraarterial, intranodal, intralymphatic, intraperitoneal, intralesional, intraprostatic, intravaginal, intrarectal, topical, intrathecal, intramuscular, intravesicular, and/or subcutaneous administration and more particularly by oral, intravenous, intradermal, intraarterial, intranodal, intralymphatic, intraperitoneal, intralesional, intraprostatic, intravaginal, intrarectal, intrathecal, intramuscular, intravesicular, and/or subcutaneous injection.

(iv) Methods of Use. Methods disclosed herein include treating subjects (humans with preexisting inflammation) with compositions disclosed herein. Treating subjects includes delivering therapeutically effective amounts. Therapeutically effective amounts include those that provide effective amounts, prophylactic treatments and/or therapeutic treatments. Therapeutically effective amounts within the current disclosure are high doses of NAD precursors.

An “effective amount” is the amount of a composition necessary to result in a desired physiological change in the subject. Effective amounts are often administered for research purposes. Effective amounts disclosed herein can cause a statistically-significant effect in an in vitro assay relevant to the assessment of a condition's development or progression.

A “prophylactic treatment” includes a treatment administered to a subject who does not display signs or symptoms of a condition to be treated or displays only early signs or symptoms of a condition to be treated such that treatment is administered for the purpose of diminishing or decreasing the risk of developing the condition further. Thus, a prophylactic treatment functions as a preventative treatment against a condition.

A “therapeutic treatment” includes a treatment administered to a subject who displays symptoms or signs of a condition to be treated and is administered to the subject for the purpose of diminishing or eliminating those signs or symptoms of the condition. The therapeutic treatment can reduce, control, or eliminate the presence or activity of the condition and/or reduce control or eliminate side effects of the condition.

Function as an effective amount, prophylactic treatment or therapeutic treatment are not mutually exclusive, and in particular embodiments, administered dosages may accomplish more than one treatment type.

In particular embodiments, therapeutically effective amounts provide anti-inflammatory effects in human subjects. Anti-inflammatory effects can be observed through increased PBMC oxygen consumption rates and/or reduced or resolved inflammation markers.

Inflammation markers include, for example, elevated expression of NLRP3, IL6, IL1β, IL18, and/or TNFα. A reduced or resolved inflammation marker can be observed as a decrease in PBMC proinflammatory marker production following administration of a high dose regimen as described herein and in comparison to a previous level observed by the PBMCs in the same subject. A therapeutically effective amount can also be observed through comparisons to a relevant baseline or reference level, as described above in relation to preexisting inflammation marker profiles.

PBMC oxygen consumption rates can be measured by a real-time cell metabolic analyzer, such as the Seahorse XFe analyzer. An anti-inflammatory effect will increase PBMC oxygen consumption rates during maximal stimulated conditions by at least 20%, at least 30%, at least 40% or at least 50% as compared to a previous measure from the same subject. Maximal stimulated conditions include those that elicit the maximal capacity of mitochondrial respiration.

PBMC inflammatory marker production can be assessed based on protein and/or mRNA expression of NLRP3, IL6, IL1β, IL18, and/or TNFα. A reduction in the expression levels of at least one of these inflammatory marker is evidence of an anti-inflammatory effect. In particular embodiments, a reduction in the expression levels of NLRP3, IL6, IL1β, IL18, and/or TNFα is evidence of an anti-inflammatory effect. Reductions in the expression of NLRP3 can also be assessed. The reduction in expression of NLRP3, IL6, IL1β, IL18, and/or TNFα can be by at least 20%, at least 30%, at least 40% or at least 50% expression at the protein and/or mRNA level as compared to a previous measure from the same subject.

For administration, therapeutically effective amounts (also referred to herein as doses) are high dose regimens of NAD precursors. The actual dose and administration protocol for a particular subject can be determined by a physician taking into account parameters such as physical and physiological factors including target, body weight, severity of condition, type of condition, previous or concurrent therapeutic interventions, idiopathy of the subject and route of administration.

The pharmaceutical compositions described herein can be administered by ingestion, injection, inhalation, infusion, perfusion, or lavage. Routes of administration can include oral, intravenous, intradermal, intraarterial, intraparenteral, intranasal, intranodal, intralymphatic, intraperitoneal, intralesional, intraprostatic, intravaginal, intrarectal, topical, intrathecal, intramuscular, intravesicular, subcutaneous, and/or sublingual administration and more particularly by oral, intravenous, intradermal, intraarterial, intraparenteral, intranasal, intranodal, intralymphatic, intraperitoneal, intralesional, intraprostatic, intravaginal, intrarectal, topical, intrathecal, intramuscular, intravesicular, subcutaneous, and/or sublingual injection.

### (V) EXEMPLARY EMBODIMENTS

1. A method including:
identifying a human Stage D heart failure patient;
orally administering to the human Stage D heart failure patient 2000 mg/day of nicotinamide riboside (NR), wherein the 2000 mg/day is split into two 1000 mg/day doses;
obtaining peripheral blood mononuclear cells (PBMCs) from the human Stage D heart failure patient;
measuring the oxygen consumption rate (OCR) of the obtained PBMCs; and
measuring the expression level of interleukin (IL)6, IL13, and IL18 by the obtained PBMCs.
2. The method of embodiment 1, wherein the orally administering is for 9 consecutive days.
3. The method of embodiments 1 or 2, wherein the identified human Stage D heart failure patient is hospitalized.
4. A method including:
identifying a human subject with a preexisting inflammation marker profile; and
orally administering a high dose regimen of a nicotinamide adenine dinucleotide (NAD) precursor to the human subject,
   wherein the high dose regimen of the NAD precursor reduces inflammation markers in the human subject's peripheral blood mononuclear cells (PBMCs).
5. The method of embodiment 4, wherein the preexisting inflammation marker profile is based on an assessment of the human subject's PBMCs expression level of an inflammatory marker.
6. The method of embodiment 5, wherein the inflammatory marker is NLRP3.
7. The method of embodiment 6, wherein the PBMC's NLRP3 expression level is higher than a baseline level.
8. The method of any of embodiments 5-7, wherein the inflammatory marker is IL6.
9. The method of embodiment 8, wherein the PBMC's IL6 expression level is higher than 6 pg/mL.
10. The method of any of embodiments 5-9, wherein the inflammatory marker is IL1β.
11. The method of embodiment 10, wherein the PBMC's IL13 expression level is higher than 1.0 pg/mL.
12. The method of any of embodiments 5-11, wherein the inflammatory marker is IL18.
13. The method of embodiment 12, wherein the PBMC's IL18 expression level is higher than 492 pg/mL.
14. The method of any of embodiments 5-13, wherein the inflammatory marker is TNFα.
15. The method of embodiment 14, wherein the PBMC's TNFα expression level is higher than 1 pg/mL.
16. The method of any of embodiments 5-15, wherein the inflammatory marker is NLRP3, IL6, IL1β, IL18, and TNFα.
17. The method of embodiment 16, wherein the PBMC's NLRP3 expression level is higher than a baseline level, the IL6 expression level is higher than 6 pg/mL, the PBMC's IL1β expression level is higher than 1.0 pg/mL, the PBMC's IL18 expression level is higher than 492 pg/mL, and the PBMC's TNFα expression level is higher than 1 pg/mL.
18. The method of any of embodiments 4-17, wherein the reduced inflammation marker in the human subject's PBMCs is an increase in the PBMC's oxygen consumption rate as compared to the PBMC's oxygen consumption rate before administering the high dose regimen.
19. The method of any of embodiments 4-18, wherein the reduced inflammation marker in the human subject's PBMCs is a decrease in the PBMC's NLRP3 expression level as compared to the PBMC's NLRP3 expression level before administering the high dose regimen.
20. The method of any of embodiments 4-19, wherein the reduced inflammation marker in the human subject's PBMCs is a decrease in the PBMC's IL6 expression level as compared to the PBMC's IL6 expression level before administering the high dose regimen.
21. The method of any of embodiments 4-20, wherein the reduced inflammation marker in the human subject's PBMCs is a decrease in the PBMC's IL1β expression level as compared to the PBMC's IL1β expression level before administering the high dose regimen.
22. The method of any of embodiments 4-21, wherein the reduced inflammation marker in the human subject's PBMCs is a decrease in the PBMC's IL18 expression level as compared to the PBMC's IL18 expression level before administering the high dose regimen.
23. The method of any of embodiments 4-22, wherein the reduced inflammation marker in the human subject's PBMCs is a decrease in the PBMC's TNFα expression level as compared to the PBMC's TNFα expression level before administering the high dose regimen.
24. The method of any of embodiments 4-23, wherein the reduced inflammation marker in the human subject's PBMCs is an increase in the PBMC's oxygen consumption rate, and a decrease in the PBMC's expression of NLRP3, IL6, IL1β, IL18, and TNFα as compared to the PBMC's oxygen consumption rate, and NLRP3, IL6, IL1β, IL18, and TNFα expression level before administering the high dose regimen.
25. The method of any of embodiments 4-24, wherein the high dose regimen includes a dose of the NAD precursor of at least 1000 mg/day.
26. The method of any of embodiments 4-25, wherein the high dose regimen includes a dose of the NAD precursor of at least 2000 mg/day.
27. The method of embodiment 26, wherein the high dose regimen includes the 2000 mg/day dose for at least 5 days, for at least 9 days, for at least 12 days, or for at least 12 weeks.
28. The method of any of embodiments 4-27, wherein the NAD precursor is nicotinamide riboside (NR).
29. The method of any of embodiments 4-28, wherein the NAD precursor is an NR analogue including NRH triacetate, NRH triproprionate, NRH tributyrate, NRH triisobutyrate, NR+ tripentanoate, NR+ trihexanoate, NRH triethylcarbonate, NRH tribenzoate, NR+ monohexanoate, NRH monodecanoate, NRH monotetradecanoate, Nic mononucleotide (NMN), NR+ monooleate, NR+ monohexanoate, NR+ monononanoate, NR+ monododecanoate, NR+ monopentanoate, or NR+ monoundecanoate.
30. The method of any of embodiments 4-29, wherein the NAD precursor is NR chloride or a crystalline form of NR chloride.
31. The method of embodiment 30, wherein the crystalline form of NR chloride includes
3-carbamoyl-1-((2R,3R,4S,5R)-3,4-dihydroxy-5(hydroxymethy)tetrahydrofuran-2-yl)pyridin-1-ium(β-D-NR) chloride crystal,
3-carbamoyl-1-((2R,3R,4S,5R)-3,4-dihydroxy-5(hydroxymethy)tetrahydrofuran-2-yl)pyridin-1-ium(β-D-NR chloride methanolate crystal, or
3-carbamoyl-1-((2S,3R,4S,5R)-3,4-dihydroxy-5(hydroxymethyl)tetra hydrofuran-2-yl)pyridin-1-ium chloride.
   32. The method of emb any of embodiments odiment 4-31, wherein the NAD precursor is an NR analogue including O-ethyl NR (OENR), tri-O-acetyl O′-ethyl NR (TAENR), N-dimethyl NR (DMNR), or N-allyl NR (ANR).
   33. The method of any of embodiments 4-32, wherein the human subject with the preexisting inflammation marker profile has a heart condition, an autoimmune disease, cancer, diabetes, a chronic respiratory disease, stroke, obesity, or allergies.
   34. The method of embodiment 33, wherein the heart condition is Stage A, B, C, or D heart failure.
   35. The method of embodiment 34, wherein the heart condition is Stage D heart failure.
   36. The method of embodiment 33, wherein the autoimmune disease is lupus or rheumatoid arthritis.
   37. A composition including at least 1000 mg of a NAD precursor.
   38. The composition of embodiment 37, including 2000 mg of a NAD precursor.
   39. The composition of embodiments 37 or 38, wherein the NAD precursor is NR.
   40. The composition of any of embodiments 37-39, wherein the NAD precursor is an NR analogue including NRH triacetate, NRH triproprionate, NRH tributyrate, NRH triisobutyrate, NR+ tripentanoate, NR+ trihexanoate, NRH triethylcarbonate, NRH tribenzoate, NR+ monohexanoate, NRH monodecanoate, NRH monotetradecanoate, Nic mononucleotide (NMN), NR+ monooleate, NR+ monohexanoate, NR+ monononanoate, NR+ monododecanoate, NR+ monopentanoate, or NR+ monoundecanoate.
   41. The composition of any of embodiments 37-40, wherein the NAD precursor is NR chloride or a crystalline form of NR chloride.
   42. The composition of any of embodiments 37-41, wherein the crystalline form of NR chloride includes
3-carbamoyl-1-((2R,3R,4S,5R)-3,4-dihydroxy-5(hydroxymethy)tetrahydrofuran-2-yl)pyridin-1-ium(β-D-NR) chloride crystal,
3-carbamoyl-1-((2R,3R,4S,5R)-3,4-dihydroxy-5(hydroxymethy)tetrahydrofuran-2-yl)pyridin-1-ium(β-D-NR chloride methanolate crystal, or
3-carbamoyl-1-((2S,3R,4S,5R)-3,4-dihydroxy-5(hydroxymethyl)tetra hydrofuran-2-yl)pyridin-1-ium chloride.
   43. The composition of any of embodiments 37-42, wherein the NAD precursor is an NR analogue including O-ethyl NR (OENR), tri-O-acetyl O′-ethyl NR (TAENR), N-dimethyl NR (DMNR), or N-allyl NR (ANR).

(vi) Experimental Examples. Experimental Example 1. Worldwide, heart failure (HF) is among the top causes of morbidity and mortality. In the United States alone, annual direct medical expenditures for heart failure exceed $30 billion (Dickstein K, et al., Eur J Heart Fail. 2008, 10(10):933-89; Lesyuk W, et al., BMC Cardiovasc Disord. 2018, 18(1):74; Redfield M M, et al., JAMA. 2003, 289(2):194-202). It has long been postulated that a chronic sterile inflammatory state in heart failure may be involved in disease progression and may contribute to worse clinical outcomes (Mann D L. Circ Res. 2015, 116(7):1254-68; Nakayama H, and Otsu K. Biochem J. 2018,475(5):839-52). While proinflammatory cytokines such as TNFα, IL1, IL6, and IL18 have been demonstrated to have a negative-inotropic effects in various experimental models (Mann D L. Circ Res. 2002, 91(11):988-98), results of anti-TNFα clinical trials in HF were discouraging (Chung E S, et al., Circulation. 2003, 107(25):3133-40; Mann D L, et al., Circulation. 2004, 109(13):1594-602). More recently, the CANTOS Trial of anti-IL1p monoclonal antibody therapy in atherosclerosis showed promising results in the HF sub-group analysis demonstrating reduction of hospitalization or heart failure-related mortality (Everett B M, et al., Circulation. 2019, 139(10):1289-99). These conflicting results likely reflect the complexity and intricacy of cytokine-mediated mechanisms of HF. A better understanding of the molecular mechanisms by which peripheral monocytes process and respond to proinflammatory signals is imperative to the development of targeted novel therapeutics for HF.

Mitochondrial dysfunction contributes to the development of heart failure via multiple mechanisms (Zhou B, and Tian R. J Clin Invest. 2018, 128(9):3716-26). Damage-associated molecular pattern (DAMP) released from mitochondria has been implicated to activate the Toll-like receptors (TLRs)/NFκB axis of peripheral monocytes in cardiac injury, leading to cytokine production and systemic inflammation (Mann D L. Circ Res. 2015, 116(7):1254-68; Nakayama H, and Otsu K. Biochem J. 2018, 475(5):839-52; Sack M N. J Clin Invest. 2018, 128(9):3651-61). Furthermore, production of reactive oxygen species (ROS) by mitochondria has been proposed as a critical step in the activation of NLRP3 inflammasome and secretion of proinflammatory cytokines (Zhou R, et al., Nat Immunol. 2010, 11(2):136-40; Zhou R, et al., Nature. 2011, 469(7329):221-5). To date, it has not been determined whether mitochondrial dysfunction of circulating immune cells contributes to the heightened inflammatory state in heart failure patients.

Recently, nicotinamide riboside (NR), a precursor in the NAD “salvage” pathway (Houtkooper R H, et al., Endocr Rev. 2010, 31(2):194-223), has been demonstrated to enhance respiratory function, reduce mitochondrial ROS (mtROS) production, and reduce IL13 production in peripheral blood mononuclear cells (PBMCs) (Traba J, et al., J Clin Invest. 2015, 125(12):4592-600). In addition, NR was shown to reduce the production of TNFα and IL6 and upregulate anti-inflammatory molecule, adiponectin, in mouse hepatocytes (Lee H J, and Yang S J. Nutr Res Pract. 2019, 13(1):3-10) and reduce circulating of IL5 and IL6 in healthy elderly subjects (Elhassan Y S, et al., Cell Rep. 2019, 28(7):1717-28 e6). Lastly, systemic NAD repletion by NR or NMN has been shown to be cardioprotective in multiple murine models of cardiomyopathy (Diguet N, et al., Circulation. 2018, 137(21):2256-73; Lee C F, et al., Circulation. 2016, 134(12):883-94; Smyrnias I, et al., J Am Coll Cardiol. 2019, 73(14):1795-806). These observations raise the possibility that targeting NAD metabolism might benefit HF patients by modulating the HF-associated inflammatory state. Targeting mitochondria-mediated inflammation is distinct from targeting a specific cytokine. It has a broader coverage of inflammatory mediators, and furthermore, it unlikely cause imbalance of cytokine levels which could occur when targeting only one cytokine.

To further explore this possibility, the following was performed: 1) PBMC respiratory function and inflammatory cytokine expression were compared from patients with HFrEF (Heart Failure with Reduced Ejection Fraction) and healthy participants, 2) the mechanistic link between mitochondrial dysfunction and inflammatory activation was investigated in PBMCs, and 3) the possibility that targeting mitochondrial metabolism by increasing NAD level with NR might attenuate PBMC inflammatory activation in vitro and in patients with HFrEF was explored.

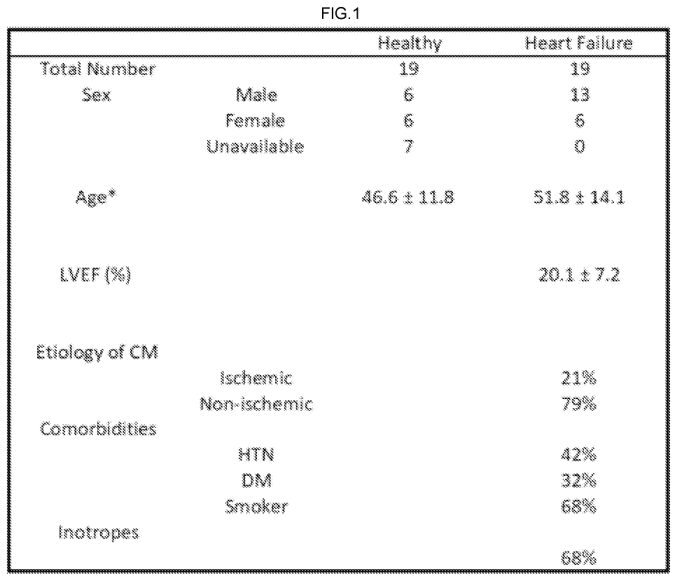

Results. Baseline characteristics of study participants. A total of 19 Stage D heart failure (HF) patients and 19 healthy participants were recruited. As shown in FIG. 1, the HF subjects were predominantly male with a mean left ventricular ejection fraction (LVEF) of 20±7%, among which 80% had non-ischemic etiology of cardiomyopathy, and 68% were on inotropic support when their blood samples were obtained. Mean age comparison between healthy and HF groups by Mann-Whitney test showed a P-value of 0.034.

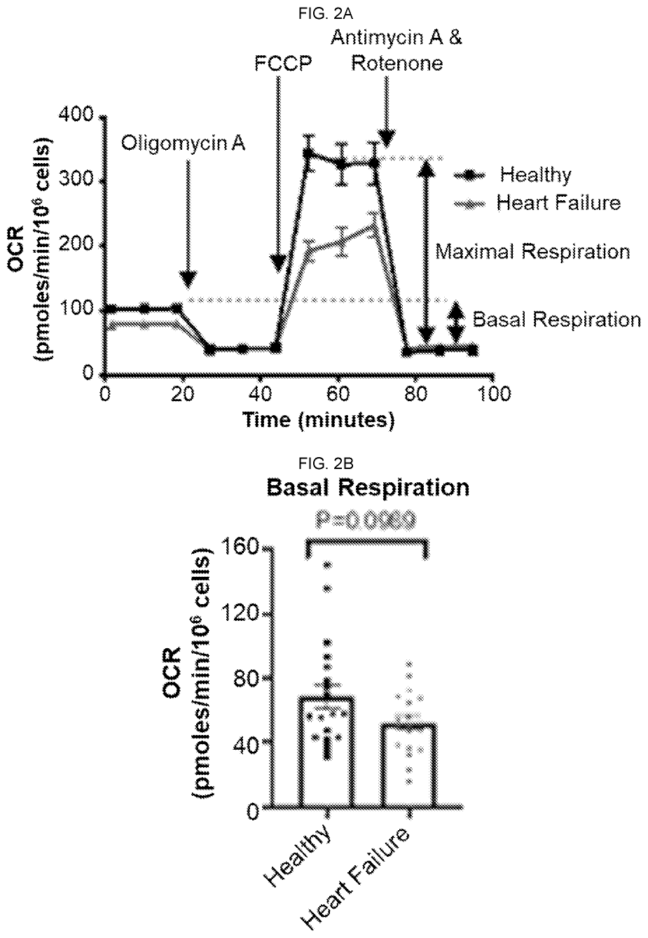

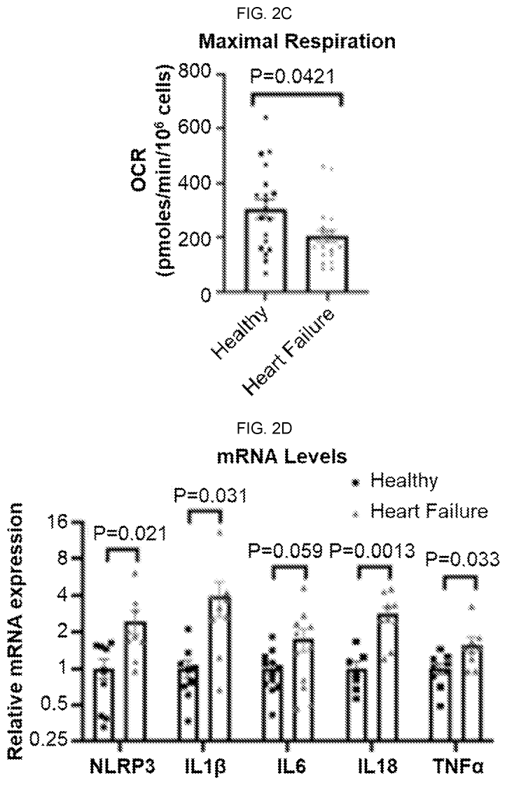

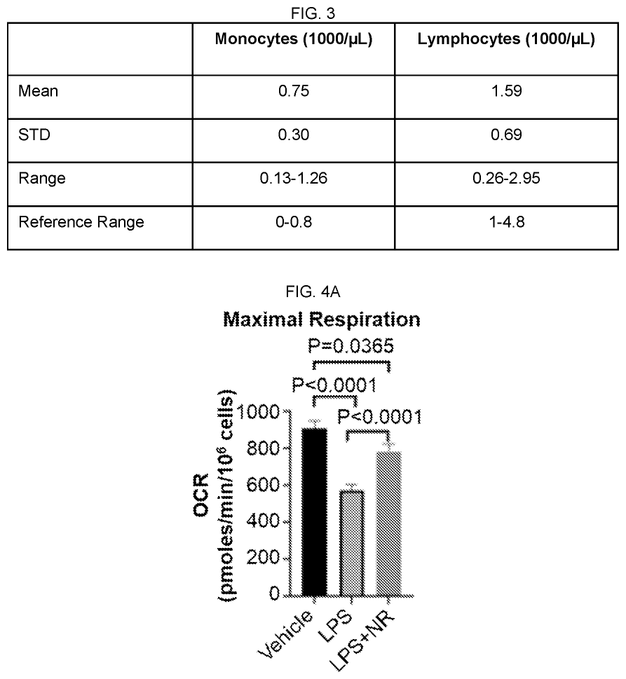

PBMCs from HFrEF patients showed reduced respiratory capacity and elevated proinflammatory cytokine gene expression. Purified PBMCs from study participants were subjected to the standard Seahorse Mito Stress Test (FIG. 2A) (Nicholls D G, et al., J Vis Exp. 2010(46)). Mean PBMC basal respiration (OCR) trended lower in HF than healthy subjects though not reaching statistical significance. (FIG. 2B). The FCCP-induced maximal OCR was significantly lower in HF patients as compared to healthy participants (FIG. 2C). Together, these findings suggest that PBMC respiratory capacity is impaired in HFrEF. Furthermore, consistent with previous reports that HFrEF is associated with a proinflammatory state, it was found mRNA levels of NLRP3, a key component of the inflammasome in monocytes and macrophages, as well as proinflammatory cytokines (IL1β, IL18, TNFα) were significantly higher in Stage D HFrEF patients as compared to healthy participants. IL6 showed a similar trend but did not reaching statistical significance (FIG. 2D). Although HF subjects had slightly higher monocyte-to-lymphocyte ratio in the PBMCs (FIG. 3), it unlikely accounted for the reduced maximal respiration or increased cytokine expression. It has been shown that basal and maximal OCR per pg protein are comparable between healthy monocytes and lymphocytes (Chacko B K, et al., Lab Invest. 2013, 93(6):690-700) and monocytes are roughly 2 times larger in volume than lymphocytes (Chapman E H, et al., J Clin Pathol. 1981, 34(10):1083-90; Zipursky A, et al., Blood. 1976, 48(3):361-71). Thus, a higher fraction of monocytes in PBMCs would increase rather than decrease OCR. Moreover, the IL1p, IL6, and TNFα productions in response to LPS simulation were shown comparable between monocytes and PBMCs (Schildberger A, et al., Mediators Inflamm. 2013, 2013:697972).

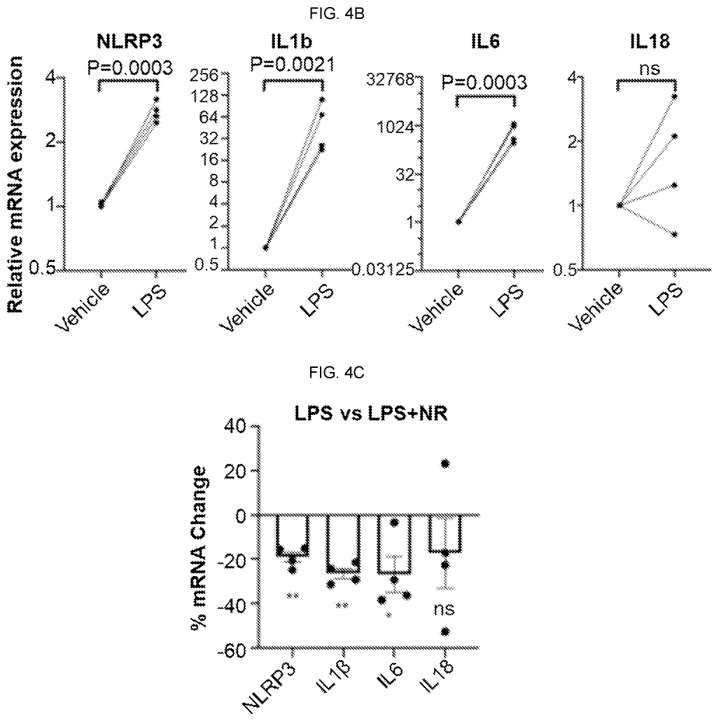

Mitochondrial DAMP induces PBMC respiratory impairment and inflammatory cytokine gene expression, which can be attenuated by inhibition of the NLRP3 inflammasome axis. To explore the potential cellular mechanisms linking mitochondrial respiratory function and cytokine production, in vitro models were created to mimic the proinflammatory state in HF. PBMCs from healthy participants were first treated with lipopolysaccharide (LPS), an endotoxin known to trigger proinflammatory activation in peripheral monocytes. 4-hours of LPS treatment resulted in a sharp decline in PBMC maximal OCR (FIG. 4A) as well as increases in mRNA expression for NLRP3 and proinflammatory cytokines (FIG. 4B), most notably, IL6.

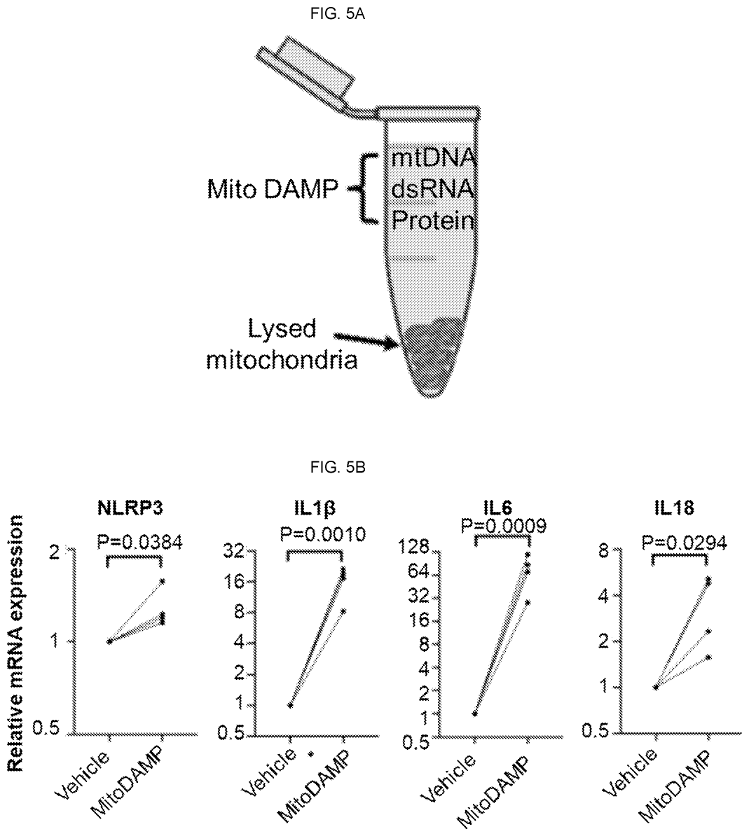

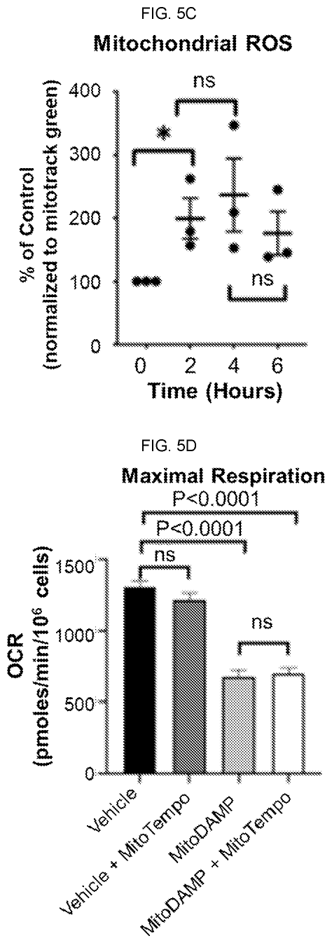

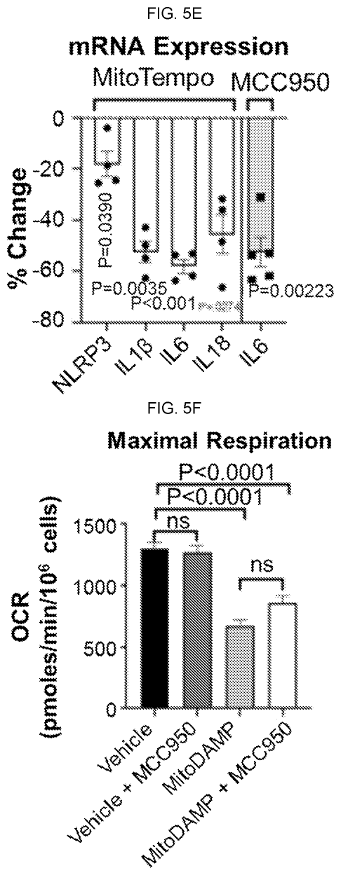

Mitochondrial content, by virtue of its evolutionary origin, can elicit an immunogenic response independent of heart failure status (Manfredi A A, and Rovere-Querini P. N Engl J Med. 2010, 362(22):2132-4). It was recently reported that plasma levels of mitochondrial DNA is elevated in heart failure patients (Dhondup Y, et al., J Card Fail. 2016, 22(10):823-8). To test whether the release of DAMP from damaged mitochondria could elicit a sterile inflammatory state in HF, MitoDAMP was extracted by lysing mitochondria isolated from myocardial tissue of end-stage heart failure patients undergoing left ventricular assist device (LVAD) surgeries (FIG. 5A). Similar to LPS, MitoDAMP treatment of healthy PBMCs induced a marked elevation of proinflammatory cytokine gene expressions (FIG. 5B).

As shown in FIG. 5C, MitoDAMP treatment resulted in a surge of mtROS production within 2 hours, which persisted for 6 hours. Mitochondrial maximal OCR was significantly impaired by MitoDAMP (FIG. 5D). mtROS generation has been reported to be one of the key activators of the NLRP3 inflammasome (Kelley N, et al., Int J Mol Sci. 2019, 20(13)), thus the role of NLRP3 inflammasome in connecting respiratory impairment and cytokine production was tested. Treating the PBMCs with MitoTempo, a nitroxide-based mitochondrial-specific ROS scavenger, in the presence of MitoDAMP, did not change maximal OCR (FIG. 5D) but markedly attenuated cytokine gene expressions (FIG. 5E). Moreover, treatment with MCC950, a specific inhibitor of the NLRP3 inflammasome, resulted in a similar attenuation of the MitoDAMP-induced surge on IL6 gene expression (FIG. 5E), while maximal OCR was marginally improved (FIG. 5F). These findings suggest that MitoDAMP-induced mtROS production and NLRP3 activation contributes to proinflammatory cytokine gene expression in PBMCs. However, inhibition of either mechanism alone appears to minimally affects maximal OCR, suggesting that mtROS generation is downstream of respiratory impairment.

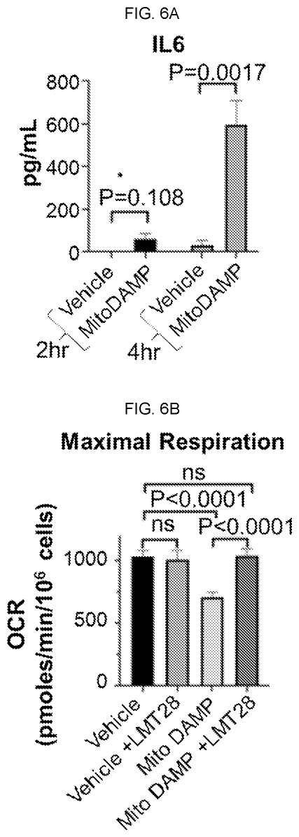

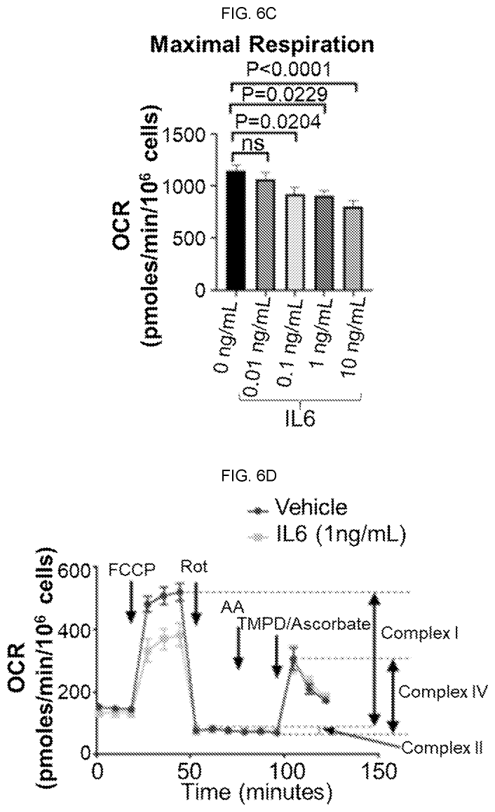

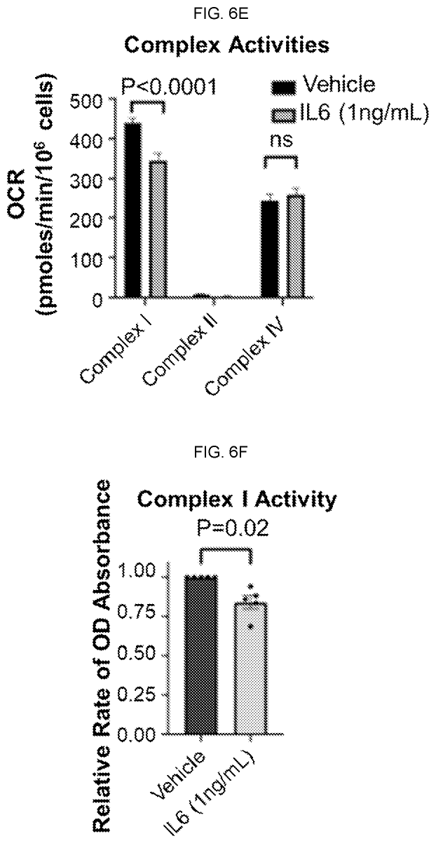

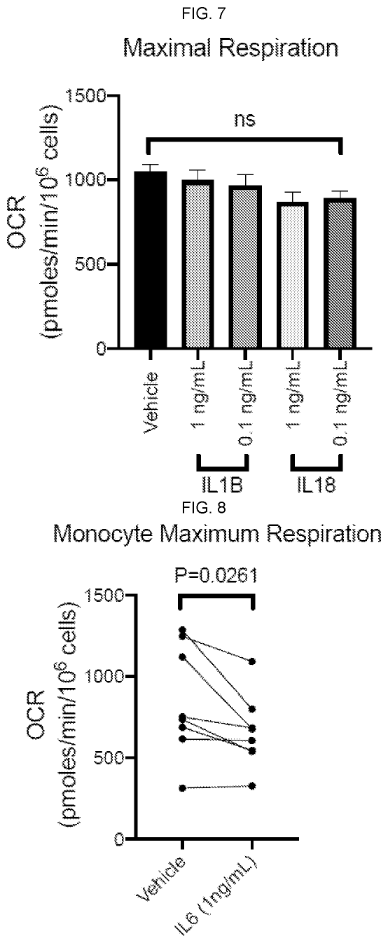

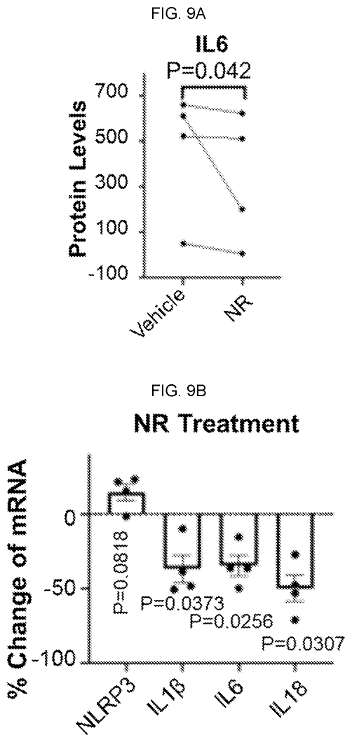

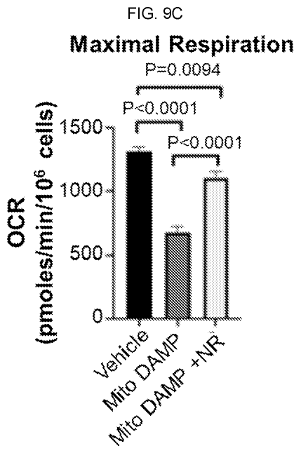

Secreted IL6 impairs mitochondrial respiration by reducing Complex I activity. Among the cytokines studied in the in vitro experiments, increases in gene expression levels in response to proinflammatory stimuli were consistently highest for IL6. Further, MitoDAMP stimulation of PBMCs resulted in a surge of secreted IL6 protein within 4 hours (FIG. 6A). It was therefore hypothesized that the MitoDAMP-induced IL6 secretion mediated the observed mitochondrial respiratory impairment in an autocrine fashion. Concurrent treatment with LMT28, an inhibitor of IL6 receptor B (GP130), prevented the MitoDAMP-induced decline in maximal OCR (FIG. 6B). Conversely, treating healthy PBMCs with recombinant IL6 resulted in a dose-dependent decline of maximal OCR (FIG. 6C). A significant reduction of maximal OCR by IL6 was observed at concentrations as low as 0.1 ng/mL, which is comparable to the plasma concentrations of IL6 during acute cardiac decompensation (Suzuki H, et al., Int J Cardiol. 2005, 100(3):415-20), as well as with the level of secreted IL6 achieved at 4 hours following Mito-DAMP stimulation in the in vitro experiments (FIG. 6A). Consistently, treatment of IL6 decreased the maximal OCR of monocytes isolated from healthy subjects (FIG. 7). In contrast, recombinant IL1β and IL18 treatments did not result in a significant decrease in PBMC maximal OCR (FIG. 8). Together, these results suggest that autocrine activation of IL6 is a key mediator of MitoDAMP-induced respiratory impairment.

Next, the end-effector of IL6-induced reduction of maximal OCR was searched for. To this end, changes in OCR in response were compared to various electron transport chain (ETC) complex inhibitors in sequence in order to identify those complex(es) whose activity was affected by IL6 treatment. As shown in FIG. 6D, complex I inhibition by rotenone virtually eliminated all FCCP-induced OCR such that subsequent inhibition of Complex III by Antimycin A only resulted in minimal OCR reduction, suggesting Complex II was a minor contributor to the overall ETC activity in the system. Moreover, when TMPD and ascorbate were added to the system as electron donors for cytochrome C, which drives OCR through Complex IV, no difference was observed in OCR between vehicle- and IL6-treated PBMCs. These results suggest that IL6 affected function of Complex I function, but not of Complexes II or IV. The contributions of each Complex to the OCR in the assay system is summarized in FIG. 6E. Consistent with these findings, direct measurement of Complex I activity showed that IL6 treatment reduced Complex I enzymatic activity (FIG. 6F). Together, these results suggest that IL6 reduces mitochondrial respiration via reduction of Complex I activity.

Increasing NAD⁺ levels with Nicotinamide riboside (NR) suppresses proinflammatory response in PBMCs in vitro. Based on the recent observations that NAD-augmentation by NR enhances respiratory function and reduces mtROS and IL13 productions in PBMCs (Traba J, et al., J Clin Invest. 2015, 125(12):4592-600), it was hypothesized that NR may ameliorate the reductions in maximal OCR and cytokine production seen with MitoDAMP stimulation. As shown in FIGS. 9A and 9B, concurrent NR treatment of healthy PBMCs in the presence of MitoDAMP resulted in a reduction of IL6 secretion and proinflammatory cytokine gene expressions, respectively. Importantly, NR treatment partially prevented the MitoDAMP-induced decline of maximal OCR (FIG. 9C). A similar effect of NR in enhancing maximal OCR and reducing proinflammatory cytokine gene expressions is also observed in LPS-treated PBMCs (FIG. 4C).

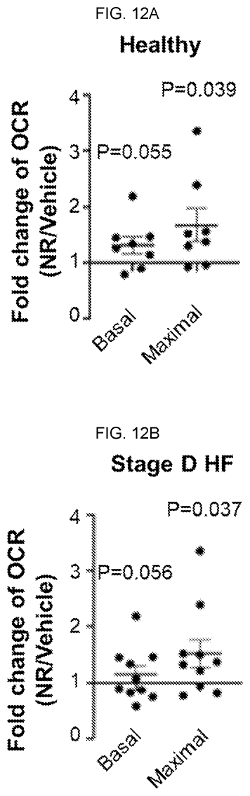

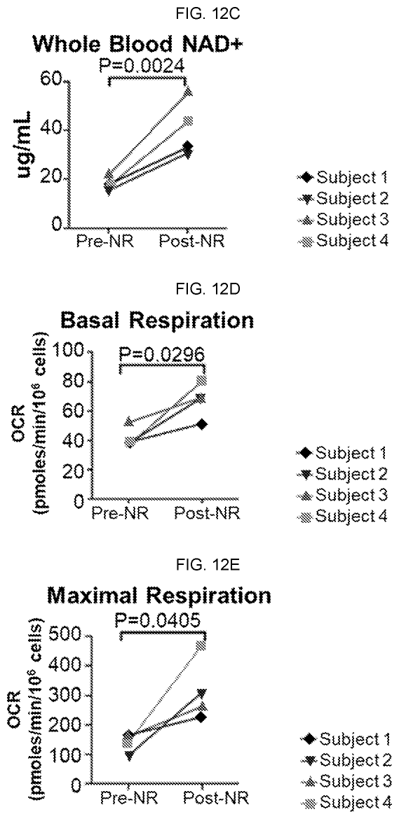

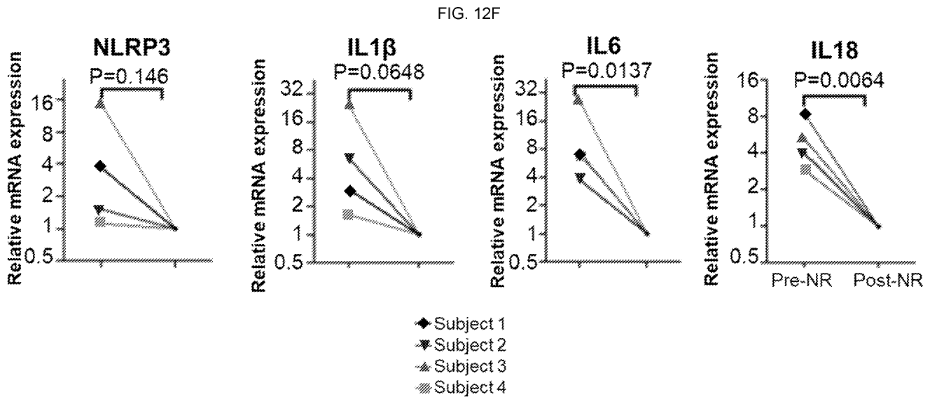

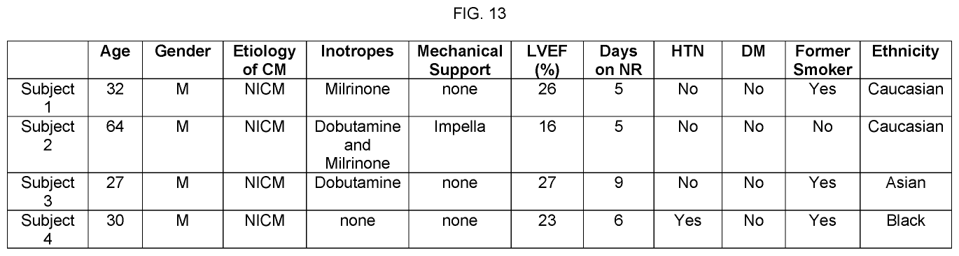

NR enhances mitochondrial respiration and reduces proinflammatory cytokine production in human heart failure. The effect of NR on the PBMCs of heart failure patients was subsequently tested in the absence of MitoDAMP. Incubation with NR in vitro increased the basal and maximal OCR in the PBMCs of both healthy and HF subjects (FIGS. 12A and 12B, respectively). However, reductions of NLRP3 and proinflammatory cytokine gene expressions in HF PBMCs were minimal (FIG. 11), indicating that a 4-hour exposure to NR may be insufficient to revert the inflammatory state. Taking these observations to the bedside, Stage D HF patients were enrolled to take NR orally for 5-9 days (FIG. 12), and blood samples were obtained pre- and post-NR administration. As shown in FIG. 12C, oral NR administration resulted in increases of whole blood NAD⁺ levels, as previously seen in healthy subjects (Airhart S E, et al., PLoS One. 2017, 12(12):e0186459). Furthermore, NR treatment resulted in a consistent enhancement of both PBMCs basal and maximal OCR (FIGS. 12D and 12E), and a up to 30-fold reduction of proinflammatory cytokine gene expressions (FIG. 12F). Background information of the study subjects can be found in FIG. 13. No adverse effect associated with NR were observed among the 5 participants.

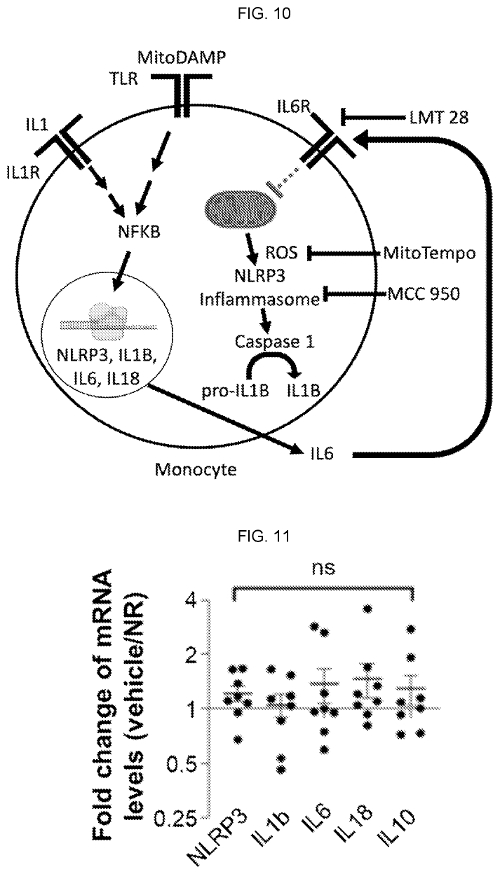

Discussion. This study demonstrates a critical role of mitochondrial function in the inflammatory activation of peripheral immune cells in HFrEF patients. It was found that proinflammatory molecules, e.g. damage associated molecular pattern released from dysfunctional mitochondria, likely via the Toll-like receptor cascade, triggers increased expressions of proinflammatory cytokines, particularly IL6. Without being bound by theory, secreted IL6 appears to feed back in an autocrine manner to impair mitochondrial respiration by inhibiting Complex I activity and inducing mitochondrial ROS production, resulting in assembly of the NLRP3 inflammasome and increased secretion of proinflammatory cytokines such as IL1β and IL18 (Zhou R, et al., Nature. 2011, 469(7329):221-5; Kelley N, et al., Int J Mol Sci. 2019, 20(13); Heid M E, et al., J Immunol. 2013, 191(10):5230-8; Sorbara M T, and Girardin S E. Cell Res. 2011, 21(4):558-60). Secreted IL1β can feedback to further potentiate the NFKB axis, resulting in a vicious cycle (Dinarello C A. Immunol Rev. 2018, 281(1):8-27). (FIG. 10)

Based on the results of the in vitro experiments, it is speculated that the effect of NR represents an upstream mechanism which preserves mitochondrial respiration and reduces ROS production in the presence of an inflammatory trigger. On the other hand, mtROS and NLRP3 are downstream of the mitochondrial dysfunction; therefore, their inhibitions do not result in improvement of maximal OCR.

It has been demonstrated that circulating IL6 is upregulated in both acute myocardial infarction and chronic heart failure; the former was thought to be cardioprotective, but if left-unchecked, chronic IL6 elevation can result in maladaptive hypertrophy and reduced LVEF (Terrell A M, et al., Shock. 2006, 26(3):226-34; Wollert K C, et al., J Biol Chem. 1996, 271(16):9535-45), potentially perpetuating heart failure. To date, there has not been a randomized clinical trial antagonizing IL6 during post-MI remodeling or in chronic heart failure. The present study suggests that IL6 assumes an important signaling role connecting mitochondrial function and inflammation in peripheral immune cells. In cultured adipocytes, in vitro IL6 treatment resulted in a decrease of mitochondrial membrane potential, cellular ATP production, an increase in intracellular ROS, and a reduction of respiratory reserve capacity (Ji C, et al., J Bioenerg Biomembr. 2011, 43(4):367-75). On the contrary, IL6 was shown to hyperpolarize mitochondrial inner membrane in CD4 cells (Yang R, et al., Elife. 2015, 4). While the exact mechanism remains elusive, there has been evidence suggesting that a small fraction of STAT3, a downstream effector of the IL6R axis, localizes in the mitochondria to regulate the electron transport chain and ATP production (Yang R, and Rincon M. Int J Biol Sci. 2016, 12(5):532-44). Whether mitochondrial STAT3 is responsible for inhibiting complex I activity during chronic sterile inflammatory states, such as heart failure, is currently underexplored.

Recently, there have been tremendous interest in agents that increase cellular NAD as potential therapeutics for heart failure. Multiple studies demonstrated that augmenting NAD level is cardioprotective in animal models of cardiomyopathy induced by pressure overload (Diguet N, et al., Circulation. 2018, 137(21):2256-73; Lee C F, et al., Circulation. 2016, 134(12):883-94; Smyrnias I, et al., J Am Coll Cardiol. 2019, 73(14):1795-806), iron-overload (Xu W, et al., Cell Rep. 2015, 13(3):533-45), or genetic mutations (Lee C F, et al., Circulation. 2016, 134(12):883-94; Vignier N, et al., Hum Mol Genet. 2018, 27(22):3870-80). Most of those studies focus on the effect of NAD in the myocardium, such that boosting myocardial NAD⁺ leads to activation of NAD-dependent deacetylases (i.e. Sirtuins) or polyADP-ribosylases. Increasing cardiac NAD levels by pharmacological or genetic approaches has been shown to improve myocardial mitochondrial function and energy metabolism partly via the activity and downstream targets of mitochondrial-specific Sirtuins (Diguet N, et al., Circulation. 2018, 137(21):2256-73; Lee C F, et al., Circulation. 2016, 134(12):883-94; Tang X, et al., Clin Sci (Lond). 2017, 131(16):2063-78; Walker M A, and Tian R. Circulation. 2018, 137(21):2274-7).

Results from this study suggests that NAD augmentation may be protective against cardiomyopathy by reducing systemic inflammation through inhibition of proinflammatory activation of circulating immune cells which, in turn, allows the system to break from the vicious cycle that perpetuates the disease. Mechanistically, NAD⁺ repletion by NR has been shown to reduce mtROS production across tissue types (Traba J, et al., J Clin Invest. 2015, 125(12):4592-600; Hong G, et al., Free Radic Biol Med. 2018, 123:125-37; Massudi H, et al., Redox Rep. 2012, 17(1):28-46; Schondorf D C, et al., Cell Rep. 2018, 23(10):2976-88; Zhang Q, et al., J Transl Med. 2018, 16(1):313), which suppresses NLRP3 inflammasome/caspase I axis and secretions of active IL1β and IL18 (FIG. 10) (Kelley N, et al., Int J Mol Sci. 2019, 20(13)). In addition, NAD⁺-dependent activities of SIRT1 and SIRT2 in the cytosol have been found to inhibit transcription factor NFKB, resulting in blunted proinflammatory cytokine gene expression (Rothgiesser et al., J Cell Sci. 2010, 123(Pt 24):4251-8; Yang H, et al., PLoS One. 2012, 7(9):e46364; Yeung F, et al., EMBO J. 2004, 23(12):2369-80). On the other hand, there is evidence that the NAD⁺-consuming enzyme CD38 is upregulated in human monocyte-derived macrophages during inflammation, and that CD38 promotes cytokine secretion (Amici S A, et al., Front Immunol. 2018, 9:1593).

This study is the first to examine whether NAD augmentation has anti-inflammatory effects in the context of human heart failure.

Methods. Study Participants. For baseline Seahorse Standard Mito Stress Test, 19 healthy (i.e. with no history of acute or chronic disease) participants and 19 hospitalized Stage D HFrEF patients were enrolled into the study. In the HF group, those requiring temporary mechanical support, such as Impella, intra-aortic balloon pump (IABP), or extracorporeal membrane oxygenation (EMCO) were excluded.

For experiments associated with the oral NR administration (FIGS. 12C-12F), 5 hospitalized Stage D HF patients undergoing advanced heart failure therapy evaluations were put on escalating doses of NR (250 mg twice a day for Day 1, 500 mg twice a day for Day 2, and 1000 mg twice a day from Day 3 on) for 5-9 days. Fasting blood samples were obtained for PBMC isolation at baseline and post-NR administration. Subject 5 was withdrawn due to a change in clinical course deemed to be independent from NR administration.

The investigation conformed with the principles outlined in the Declaration of Helsinki.

PBMC and Monocyte isolation. 30-60 ml of fasting blood was collected into EDTA-containing Vacutainers (BD Mfr #364606). Blood then was diluted (1:1) with RPMI medium (Corning; Cat #17-105-CV) and applied to Histopaque gradient medium (Sigma-Aldrich; Cat #10771) using SepMate-50 (STEMCELL Technologies), and centrifuged at 1200 g for 10 minutes. Top layer contained the enriched peripheral blood mononuclear cells (PBMCs) which were collected and followed by centrifugation at 300 g for 10 minutes. The pellet was resuspended with ACK lysis buffer (Gibco; Cat. A1049201) and incubated at room temperature for 5 minutes to remove residual red blood cells. Next, the enriched PBMCs were washed twice with RPMI medium. All PBMC samples were subjected to the baseline Seahorse Mito Stress Test. When available, the remaining cells were used for cytokine mRNA quantitative PCR and in vitro assays. For monocyte isolation, a magnetic bead-based negative-selection monocyte isolation kit (Miltenyi Biotec, 130-096-537) was used per the manufacturer's instructions. Briefly, antibody-conjugated magnetic bead solution was added to isolated PBMCs resuspended in RPMI containing 0.5% BSA and incubated for 20 minutes at 4° C., followed by magnetic column binding and elution. The procedure was repeated once to improve the purity of monocytes. The monocytes were washed once with RPMI prior to subsequent experiments.

Seahorse Mito Stress Test. Oxygen consumption rate (OCR) of freshly isolated PBMCs was assessed using a Seahorse XFe24 analyzer (Agilent Technologies). Briefly, purified PBMCs were resuspended in Seahorse base medium (Agilent Technologies, supplemented with 1 mM pyruvate, 2 mM glutamine, and 10 mM glucose, pH 7.4), and added 10⁶ cells/well at equal volume of 500 μL in a Seahorse 24-well plate. The cells were maintained in a non-CO₂ incubator and allowed to settle for 30 minutes at 37° C. Next, the plate was centrifuged at 40 g for 10 minutes at room temperature without application of the brake to help cells attach to the bottom of the plate. Drugs used in the assay: 5 μM oligomycin A (Sigma-Aldrich; Cat. 75351), 3 μM Trifluoromethoxy carbonylcyanide phenylhydrazone (FCCP) (Sigma-Aldrich; Cat. C2920), 1 μM Rotenone (Sigma-Aldrich; Cat. A8674), 1 μM Antimycin A (Sigma-Aldrich; Cat. R8875), 0.5 mM TMPD (Sigma-Aldrich; Cat. T7394) and 10 mM Ascorbate (Sigma-Aldrich; Cat. 95209).

PBMC and Monocyte Culture. Freshly isolated PBMCs or Monocytes from healthy or HF subjects were resuspended with RPMI medium plus 10% heat inactivated FBS. PBMCs (10⁶ cells/well) were seeded in Seahorse 24-well plates and incubated at 37° C. in CO₂ incubator with various proinflammatory triggers (Vehicle, MitoDAMP, LPS) and compounds (NR, MCC 950, MitoTempo, or LMT-28). After 4 hours incubation, the plate was centrifuged at 40 g for 10 minutes at room temperature without application of the brake to help cells attach to the bottom of the plate. The cells were washed twice and then brought up to 500 μL using Seahorse base medium (Agilent Technologies, supplemented with 1 mM pyruvate, 2 mM glutamine, and 10 mM glucose, pH 7.4).

MitoDAMP Isolation. Human heart tissue samples (100-200 mg) from the left ventricular apex were collected at the time of left ventricular assist device (LVAD) implantation when tissue from the LVAD core became available. Fresh tissue was stored in ice-cold sterile PBS prior to mitochondria isolation per the protocol described in Lee C F, et al., Circulation. 2016, 134(12):883-94. The purified mitochondria were resuspended in 200 μL of MSE buffer (Lee C F, et al., Circulation. 2016, 134(12):883-94), and subsequently stored in −80° C. Mitochondria from 6 donors were pooled and lysed by freeze (in liquid nitrogen) and thaw for 3 times, followed by sonication at 30% intensity with 3-sec-on and 3-sec-off for 3 cycles. The lysed mitochondria were then spun down at 10,000 RPM for 10 minutes. Protein concentration of the supernatant (MitoDAMP) was determined by BCA kit. The supernatant was aliquoted and stored at −80° C. for future use. For all MitoDAMP stimulation assays, the final protein concentration of MitoDAMP was 0.5 mg/mL (FIG. 5A).

IL6 ELISA. Isolated PBMCs (4×10⁶ cells per sample) from healthy or HF participants were resuspended in RPMI medium containing 10% heat-inactivated FBS and treated with various proinflammatory triggers (Vehicle, MitoDAMP, LPS) and compounds (NR, MCC 950, MitoTempo, or LMT-28) to final PBMC concentrations of 10×10⁶ cells per mL. See below for detailed descriptions of the reagents. The samples were incubated in Eppendorf tubes at 37° C. in a 5% CO₂ incubator with caps open for times designated in the brief description of drawings. The cultured PBMC samples were centrifuged at 8,000 RPM at room temperature. The supernatant was used for ELISA per the manufacture protocol using R&D Systems human IL6 kit (Cat #DY206-05). The cell pellets were stored in −80° C.

RNA Isolation and Quantitative PCR analysis. Total RNA was isolated from frozen PBMC pellets using Trizol (Invitrogen) per the manufacturer's instructions, and cDNA was synthesized using iScript™ Reverse Transcription Supermix (Bio-rad) per the manufacturer's instructions. Real-time PCR was performed in the Corbett rotor gene 6000 real-time PCR machine using iQ™ SYBR® Green Supermix (Bio-rad) and expression levels of the indicated genes were calculated using the ΔΔCt method. Primer sequences were as follows:

NLRP3 forward:
(SEQ ID NO: 1)
GTGTTTCGAATCCCACTGTG;
reverse:
(SEQ ID NO: 2)
TCTGCTTCTCACGTACTTTCTG;
IL1β forward:
(SEQ ID NO: 3)
ATGCACCTGTACGATCACTG;
reverse:
(SEQ ID NO: 4)
ACAAAGGACATGGAGAACACC;
IL6 forward:
(SEQ ID NO: 5)
CCACTCACCTCTTCAGAACG;
reverse:
(SEQ ID NO: 6)
CATCTTTGGAAGGTTCAGGTTG;
TNFα forward:
(SEQ ID NO: 7)
ACTTTGGAGTGATCGGCC;
reverse:
(SEQ ID NO: 8)
GCTTGAGGGTTTGCTACAAC;
IL18 forward:
(SEQ ID NO: 9)
CATTGACCAAGGAAATCGGC;
reverse:
(SEQ ID NO: 10)
CACAGAGATAGTTACAGCCATACC;
18S rRNA forward:
(SEQ ID NO: 11)
GTAACCCGTTGAACCCCATT;
reverse:
(SEQ ID NO: 12)
CCATCCAATCGGTAGTAGCG.

Mitochondrial ROS detection in cultured PBMCs. To detect mitochondrial ROS production, the treated PBMCs were incubated with 5 μM MitoSOX Red (Life Technologies) and MitoTracker Green (200 nM, Life Technologies) at 37° C. for 30 minutes in RPMI medium supplemented with 10% heat-inactivated FBS, and then washed twice with cold PBS before cells were analyzed by flow cytometry on a Cytek Aurora (Cytek Biosciences).

Complex I in vitro assay. This assay was performed using Abcam Complex I Enzyme Activity Microplate Assay Kit (ab109721), per the manufacturer's protocol. Briefly, following a 4-hour treatment with vehicle or IL6, 5×10⁶ PBMCs were washed with PBS and lysed by detergent for 30 minutes on ice, followed by centrifugation at 16,000 g for 20 minutes at 4° C. Supernatant was taken for protein concentration determination. 12.5 μg of total protein in a final volume of 200 μL was incubated in microplates precoated with anti-Complex I antibody for 3 hours at room temperature. Post-washing step, NADH and dye were added, and optical density (OD) was serially measured at 450 nm over 1 hour. The slope of the curve within the linear range was recorded as Complex I activity.

Whole bloodNAD⁺ assays. These were performed as previously described (Airhart S E, et al., PLoS One. 2017, 12(12):e0186459). Briefly, 50 μL of thawed blood samples were added to tubes containing 1 μg BMP as the internal standard and immediately followed by 300 μL of 4% trichloroacetic acid to precipitate proteins. Calibration standards were similarly prepared except that the matrix was 30 mg/mL BSA spiked with NAD⁺ and NMN that had been dissolved in methanol. After protein precipitation and high-speed centrifugation, the supernatants were loaded onto Costar Spin-X 0.22 μm spin filters and subject to high speed centrifugation again. The eluates were injected onto an Agilent 1100 series high performance liquid chromatograph coupled to an Agilent G1956B single-quadrupole mass spectrometer. The mass spectrometer was operated in electrospray ionization mode with positive polarity.

Reagents.

Lipopolysaccharide (LPS)—purified from Salmonella Minnesota R595, was purchased from Enzo life sciences, Cat #ALX-581-008-L001.

MCC 950—purchased from Sigma Aldrich, Cat #PZ0280-5MG, stock dissolved in RPMI to 100 μM and stored in −80° C.

MitoTempo—purchased from Sigma Aldrich, Cat #SML07375MG, stock solution dissolved in RPMI to 10 mM and stored in −80° C.

LMT-28—purchased from Sigma Aldrich, Cat #SML1628-5MG, stock solution dissolved in DMSO to 10 mM and stored in −80° C.

Human Recombinant IL6—purchase from Gibco, Ref #PHC0064, stock solution dissolved in RPMI to 1 μg/mL and stored in −80° C.

Human Recombinant IL1β and IL18—purchased from R&D Systems, Cat #201-LB and #9124-IL, respectively. Stock solutions dissolved in RPMI to 1 μg/mL and stored in −80° C.

Nicotinamide Riboside (NR). For in vitro assays, NR was supplied as a powder by the manufacturer (Niagen®, ChromaDex, Irvine, Calif.). 10 mM or 100 mM stock solutions dissolved in RPMI with 10% heat inactivated FBS were made on the day of the experiment. For oral administration, NR was supplied by the manufacturer as 250 mg capsules, and manufactured in a GMP-compliant facility according to ISO/IEC 18025:2005 standards.

Statistical Analyses: Statistical analyses were performed using GraphPad Prism 8 (Version 8.3.0). For analyses of PBMC respiratory function, the primary outcome was defined as the mean difference in basal OCR and maximal OCR (post FCCP injection), determined by Seahorse Mito Stress Test in PBMCs of healthy vs. HFrEF participants. Mean age comparison between subject groups were subjected to unpaired 2-tailed nonparametric test (Mann-Whitney test). For mRNA expression comparisons between healthy and HF participants, P-value was determined by unpaired parametric 2-tailed t-test. In vitro and in vivo pre- vs post-treatment analyses were performed by paired 2-tailed parametric t-test or 1-way ANOVA followed by post-hoc pairwise multiple comparisons, as appropriate. Error bars in FIGs. represent standard error of means (SEMs).

StudyApproval: All studies were approved by the Human Subject Division of University of Washington. For baseline PBMC measurements, blood samples from adult healthy participants were collected under IRB-approved protocol (STUDY00005599), and HFrEF patient blood and myocardial tissue samples were collected under a separate IRB-approved protocol (STUDY00002544).

For the experiments pertaining oral NR administration in HFrEF patients, the study was performed under the IRB-approved protocol STUDY0000543, ClinicalTrials.gov identifier: NCT03727646.

Experimental Example 2. Despite the use of guideline-directed medical therapies (Fiuzat M, et al., JAMA Cardiol. 2020, 5(7):757-764; Vaduganathan M, et al., JACC Heart Fail. 2020, 8:469-480), heart failure remains a major cause of morbidity and mortality in the US, affecting more than 6M individuals at an estimated annual cost of over $30M (Virani S S, et al., Circulation. 2020, 141:e139-e596). Because current medical treatments for heart failure primarily target the neurohormonal activation characteristic of this disease syndrome, novel therapeutic approaches are needed (Tian R, et al., 2019, 140:1205-1216).

Failing myocardium is characterized by a decrease in nicotinamide adenine dinucleotide (NAD) level and the ratio of NAD+ to NADH (Karamanlidis G, et al., Cell Metab. 2013, 18:239-50; Lee C F, et al., Circulation. 2016, 134:883-94; Diguet N, et al., Circulation. 2018, 137:2256-2273). These changes impact redox balance, cell death and inflammation, as well as in post-translational protein modifications important to cellular metabolic processes and energy transduction (reviewed in Zhou B and R Tian, J Clin Invest. 2018, 128:3716-3726).

Nicotinamide riboside (NR), an orally bioavailable NAD+ precursor, recently has been shown to be both well-tolerated and effective in increasing peripheral blood NAD+ levels in small, Phase I clinical trials of healthy volunteers (Trammell S A, et al., Nat Commun. 2016, 7:12948; Airhart S E, et al., PLoS One. 2017, 12:e0186459; Martens C R, et al., Nat Commun. 2018, 9:1286). Additional human studies have found NR to be well-tolerated in obese (Dollerup O L, et al., Am J Clin Nutr. 2018, 108:343-353; Dollerup O L, et al., J Clin Endocrinol Metab. 2019, 104:5703-5714) and older (Dolopikou et al., Eur J Nutr. 2020 59(2):505-515; Elhassan Y S, et al., Cell Rep. 2019, 28:1717-1728 e6) individuals. Further, anti-inflammatory effects of NR have been reported in small clinical trials (Elhassan Y S, et al., Cell Rep. 2019, 28:1717-1728 e6; Traba J, et al., J Clin Invest. 2015, 125:4592-600; Zhou B, et al., J Clin Invest. 2020, 130:6054-6063 (Experimental Example 1)).

Therefore, to test the hypothesis that oral NR supplementation may be of clinical benefit in heart failure, the present study was undertaken of NR in 30 participants with clinically-stable, HFrEF to test: 1) NR safety and tolerability (primary outcome), 2) effects of NR on whole blood NAD+ levels, incidence of on-trial abnormal laboratory values and adverse events, and effects on mitochondrial function in peripheral blood mononuclear cells (PBMCs) (secondary outcomes) and 3) exploratory endpoints of changes in functional capacity by six minute walk distance (6MWD), echocardiographically-determined left ventricular (LV) systolic function (2D-LVEF) and diastolic function (E/e′ ratio), and quality of life by the Minnesota Living with Heart Failure Questionnaire (MLHFQ).

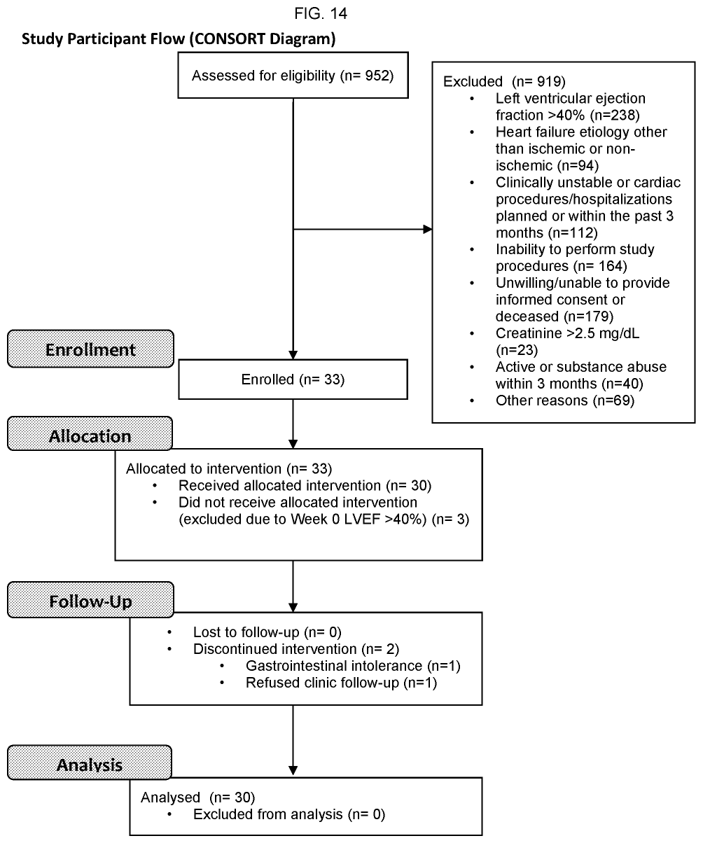

Methods. Participants. Of 952 HF patients screened, 33 participants were recruited from the Cardiology Clinic at the University of Washington Regional Heart Center. The first participant was enrolled on May 19, 2016, and the final participant completed follow-up on Mar. 11, 2019. Primary reasons for exclusion were: a) LVEF>40% (25.9%), b) unwilling or unable to provide informed consent or deceased (19.5%), c) inability to undergo study procedures, (17.8%), d) clinically unstable or cardiac procedures/hospitalizations within the past 3 months (12.2%) and e) heart failure etiology other than ischemic or non-ischemic (10.2%). Of these 33 participants, three were found at their randomization visit (Week 0) to have an LVEF that had improved to >40%, and so were withdrawn from the Trial prior to receipt of randomized therapy. Flow of participants through the Study is shown in FIG. 14.

The remaining 30 participants met all the prespecified inclusion criteria of: 1) Systolic HF (LVEF<40%, as determined by transthoracic echocardiogram) of non-ischemic or ischemic etiologies, 2) clinically stable (no cardiac procedures or hospitalizations for cardiac causes, including HF, ischemia or arrhythmia) within the prior 3 months, 3) ability to perform Study procedures (including study visits, blood draws and 6MWT), 4) willingness/ability to provide informed consent. None had any of the following exclusion criteria: 1) Heart failure with preserved ejection fraction (LVEF>40%), 2) HF etiologies other than non-ischemic or ischemic, 3) cardiac procedures, including, cardiac surgery, PCI, or cardiac device implantation, within the previous 3 months, 4) hospitalizations for cardiovascular causes, including HF, chest pain, stroke/TIA or arrhythmias within the prior 3 months, 5) inability to perform Study visits or procedures (e.g., physical inability to perform 6MWT), 6) unwillingness or inability to provide informed consent, 7) Alanine transaminase (ALT)>×3 upper limit of normal, hepatic insufficiency or active liver disease, 8) recent history of acute gout, 9) chronic renal insufficiency with serum creatinine ≥2.5 mg/dl, 10) pregnant or likely to become pregnant, 11) significant co-morbidity likely to cause death within 6 months, 12) significant history of active substance abuse within the previous 5 years, 13) current participation in a clinical trial of a study drug or intervention, and 14) prior intolerance to NR precursors, including niacin or nicotinamide. Follow-up was completed on all 30 participants. Study medication was discontinued in one participant due to gastrointestinal intolerance, but he completed all follow-up visits and procedures. The final participant refused to complete any study visits or procedures after the Week 0 (Randomization Visit), but his vital status was confirmed at the end of the Study. No participants were excluded from analysis, which followed the principal of intention-to-treat. The standardized method for characterizing adverse events employed in the UW Clinical Trials Unit is the Common Terminology Criteria for Adverse Events v3.0 (CTCAE). There were 18 protocol deviations.

All research involving human participants was approved by the University of Washington Institutional Review Board (IRB), and all clinical investigation was conducted according to the principles expressed in the Declaration of Helsinki. Written informed consent was obtained from all participants.

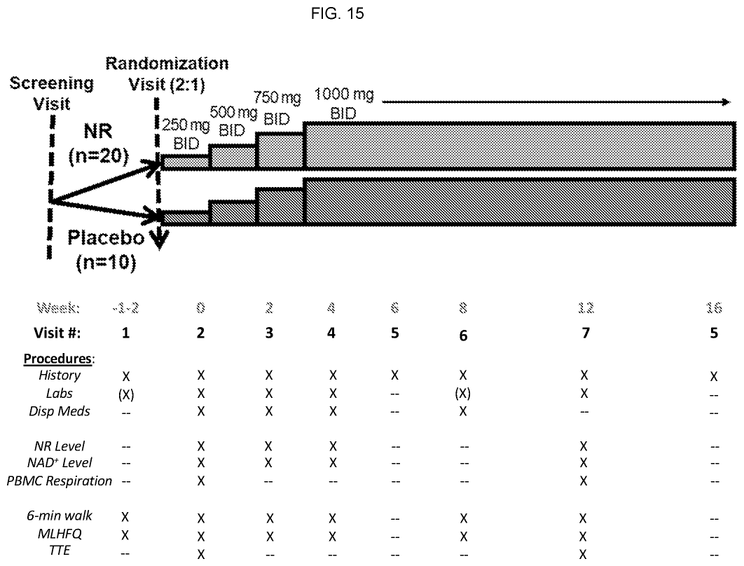

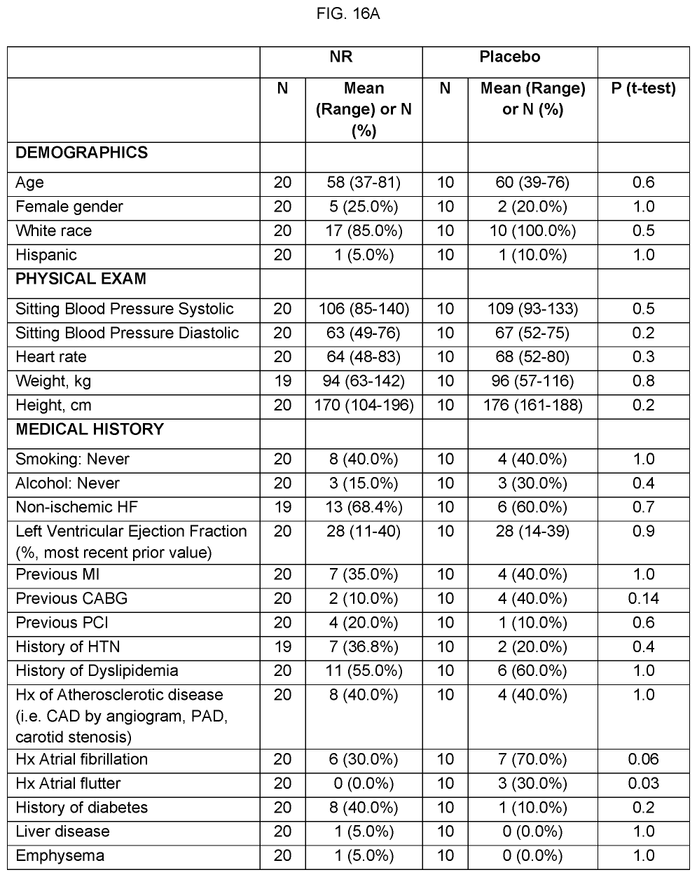

Study Design: The overall Study Design is depicted graphically in FIG. 15. Participant clinic visits, medication dispensing and data collection were performed on the Translational Research Unit (TRU) at the University of Washington (UW). At the Screening Visit, participants had a history, baseline laboratories and blood sample collection for peripheral blood mononuclear cell (PBMC) isolation and mitochondrial respiration assay (Seahorse XF®, Agilent, Santa Clara, Calif.) and assessment of 6-minute walk distance (6MWD). History included assessment with the Minnesota Living with Heart Failure Questionnaire (MLHFQ), under license from the University of Minnesota (Minneapolis, Minn.).

Participants were randomized to NR or placebo at a 2:1 allocation ratio by the University of Washington Investigational Drug Service, which assigned Study IDs and dispensed study medications.

At Week 0 (Randomization), participants underwent history, repeat 6MWD assessment and baseline transthoracic echocardiogram (FIG. 15). NR or matching placebo were started at the initial dose of 250 mg orally twice daily, then up-titrated by 250 mg twice daily each week to a final dose of 1000 mg twice daily by Week 3. Participants were continued on the 1000 mg twice daily dose until the final clinic visit on Week 12. Blood draws for study labs, including levels of NR and NAD were obtained at Weeks 0, 2, 4 and 12. Medications were dispensed at Weeks 0, 2, 4 and 8, with pill counts performed at Weeks 2, 4, 8 and 12. A telephone visit was performed at Week 6. The final echocardiogram and PBMC mitochondrial respiration assays were performed at Week 12. Study labs were performed by the UW Research Testing Service, except for NR and NAD+ assays, which were performed by the Pharmacokinetics Laboratory, Department of Pharmaceutics, UW School of Pharmacy. Procedures for blood collection and processing, as well as NR and NAD+ assay procedures, have been described previously (Airhart S E, et al., PLoS One. 2017, 12:e0186459).

Ethics. The study protocol followed UW ethical standards in accordance with the Helsinki Declaration of 1975 as revised in 1983. Study recruitment was conducted according to UW Institutional Review Board (IRB) policies, as well as Health Insurance Portability and Accountability Act (HIPAA) policies. All participants provided written, informed consent. The study was registered on www.clinicaltrials.gov (Identifier: NCT03423342).

A Study Data and Safety Monitoring Committee (DSMC) reviewed unblinded safety, tolerability, laboratory results and endpoints after Study completion by 10, 20 and 30 participants. All Study personnel remained blinded to randomized treatment assignment until after Study completion and final review by the DSMC.

Nicotinamide Riboside: Source and Authentication. NR and matching placebo were supplied by the manufacturer as 250 mg capsules (Niagen®, ChromaDex, Irvine, Calif.). NR was manufactured in a GMP-compliant facility according to ISO/IEC 18025:2005 standards. Two Certificates of Analysis provided by the manufacturer and performed on separate lots reported 99% purity of the NR preparation.

Peripheral Blood Mononuclear Cell (PBMC) Mitochondrial Function. PBMCs were isolated from freshly-collected whole blood by centrifugation in Sepmate™-50 PBMC isolation tubes (STEMCELL Technologies, Vancouver, BC, Canada; Cat. 85450). Isolated PBMCs were resuspended in Seahorse XF® medium (Agilent, Cat. 102353-100) and then plated (10⁶ cells/well) onto Seahorse® (Agilent) 24-well plates. PBMC mitochondrial respiratory function was assessed by measuring the oxygen consumption rate (OCR) at baseline and maximal stimulated conditions as described in Zhou B, et al. (J Clin Invest. 2020, 130:6054-6063 (Experimental Example 1)). Exploratory Endpoints. The following endpoints were considered exploratory, as the Study was underpowered to detect differences.

Six minute walk distance (6MWD). Any potential “training” effect (Laboratories ATSCoPSfCPF. ATS statement: guidelines for the six-minute walk test. Am J Respir Crit Care Med. 2002, 166:111-7) for 6MWD results was mitigated by performing a “training” test at the Screening Visit, while the second test, performed at the Randomization Visit was considered the Baseline test.

Echocardiography. Serial echocardiograms were performed using a standardized data collection protocol determined using the TOMTEC Arena Image-Com (TOMTEC Imaging Systems, Unterschleissheim, Germany). All echocardiograms were interpreted by a single observer, blinded to participant clinical data, randomized treatment assignment and echocardiogram temporal sequence. For evaluation of left ventricular systolic function by 2-dimensional (2-D), transthoracic echocardiography, 28 study pairs (NR=18, placebo=10) had evaluable data. For evaluation of left ventricular diastolic function by strain imaging (E/e′), 24 study pairs (NR=17, placebo=7) had evaluable data.

Minnesota Living with Heart Failure Questionnaire (MLHFQ). The MLHFQ was administered to Study participants at the Screening and Randomization visits, as well as at Study visits at Weeks 2, 3, 4, 8 and 12.

Statistical Analyses. Descriptive statistics are presented as means, standard deviations and ranges for continuous variables and as counts and percentages for categorical variables. Statistical significance of differences in patient characteristics and outcomes between NR and placebo patients were calculated using the two-sample t-test with unequal variances, the chi-squared test or Fisher's exact test (as appropriate). Normal quantile-quantile plots were used to visually assess departures from normality. Highly skewed variables were analyzed as log values. Spearman correlation quantified associations between pairs of variables. Statistical analyses were performed using R version 3.6.0 (Vienna, Austria) and GraphPad Prism 8 (Version 8.3.0). P<0.05 was used to denote statistical significance.

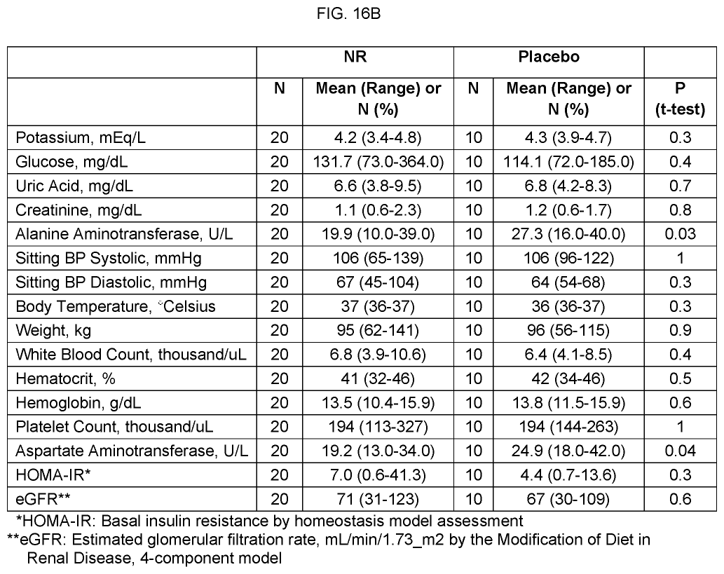

Results. Participant Characteristics. Study participants were (mean±SD) 59±8 years of age and 23% female with the following, self-reported ethnic distribution: 83% non-Hispanic white, 7% Hispanic, 7% Asian and 3% African American. HF etiology was non-ischemic in 63% and mean LVEF at baseline was 28%. Study participant characteristics by randomized group were well-matched at baseline for demographics, physical exam and medical history (FIG. 16A), as well as for baseline laboratory values (FIG. 16B).

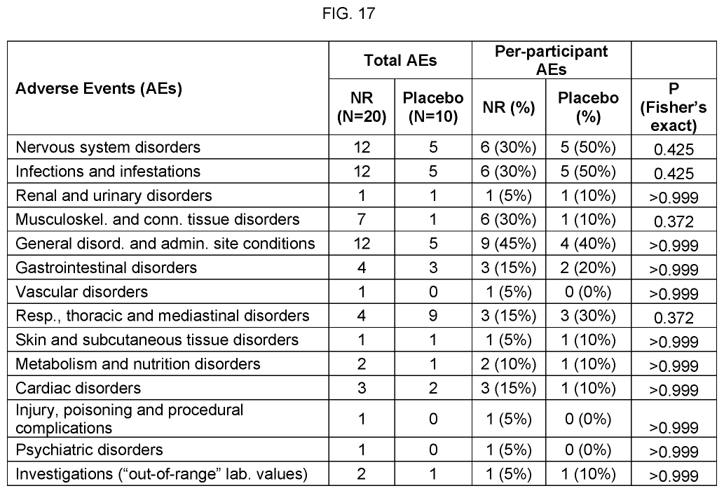

NR Safety and Tolerability. In this relatively small trial, no between-group differences were observed in treatment-emergent adverse events (AEs). On-trial a total of 97 AEs were reported, 63 in the 20-participant NR group 34 in the 10-participant placebo group. FIG. 17 summarizes Total AEs and per-participant AEs by randomized group. There was 1 SAE, a hospitalization for heart failure exacerbation and pancreatitis, in a placebo group participant.

Out-of-range lab values (classified as “Investigations” in FIG. 17) were rare, with creatinine greater than 1.5× but less than 3× baseline on 2 occasions in 1 patient randomized to NR, and ALT greater than 3× but less than 5× baseline in 1 patient randomized to placebo.

NR tolerability was high, as assessed by on-trial compliance with randomized treatments. Based on returned pill counts, percentages of dispensed capsules taken (range) were similar for the NR group: 97 (91-100)% and for the placebo group: 95 (89-100)%, p=0.15.

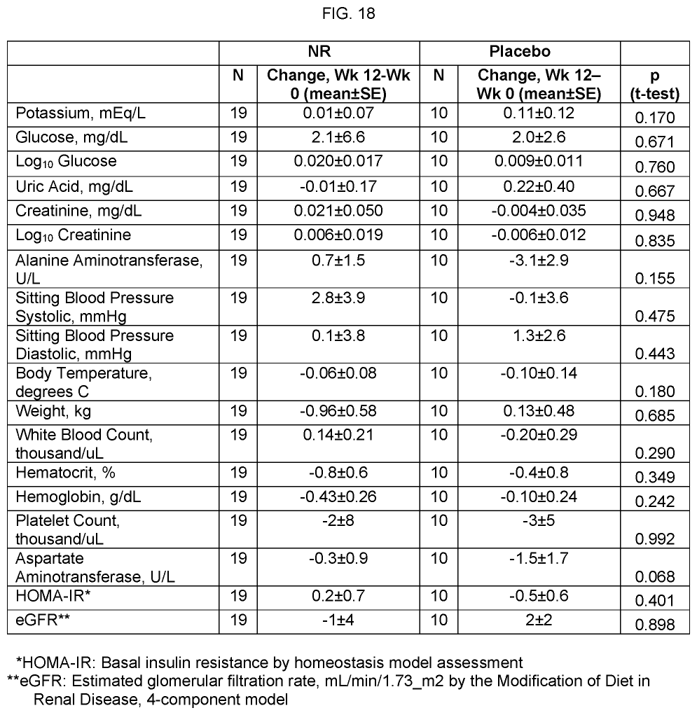

There were no significant, on-trial changes (Week 12-Week 0) in pre-specified, laboratory or clinical variables (FIG. 18).

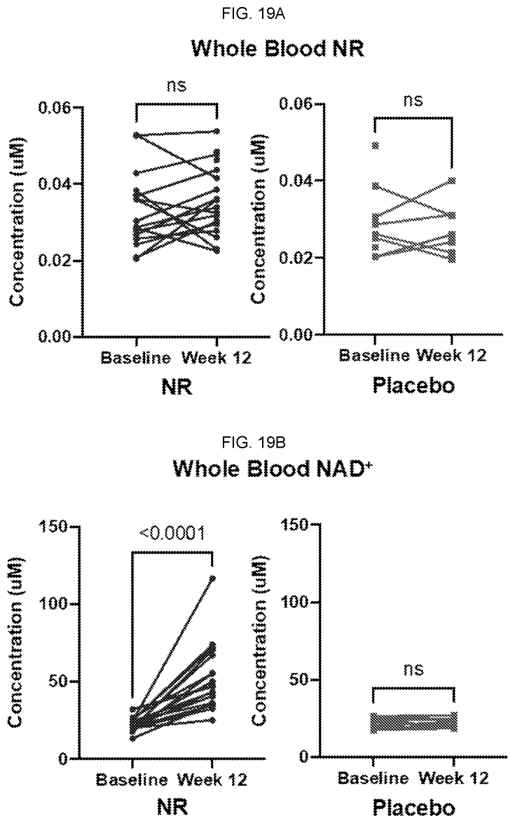

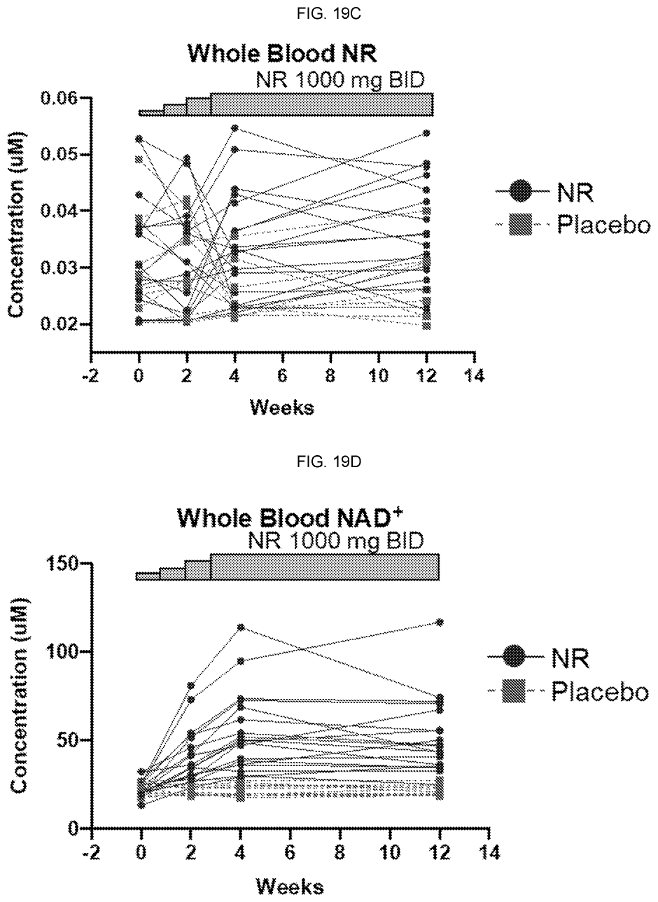

On-Trial NR and NAD+ Levels. On-trial changes in whole blood NR levels (Week 12-Week 0) were unchanged in both the NR and placebo groups (Mean±SE changes: NR group: 0.002±0.009 μM, p=0.5 vs. placebo group:0.001±0.007 p=0.8, though variability in response was high. (FIG. 19A)

Changes in whole blood NAD+ levels on-trial (Week 12-Week 0) were significantly higher in those randomized to NR (Mean+SE changes: NR group: 30±20 μM p=0.7 vs. placebo group: −0.3±2 μM, p=1e-05***). (FIG. 19B)

The blood NR level was low and variable before the daily dose was administered (FIG. 19C).

During the up-titration period, there was a dose-dependent increase of blood NAD+ levels in the NR group. NAD+ levels increased from Week 0 to Week 2 (after 1 week on NR 500 mg twice daily). NAD+ levels increased further by Week 4 (after 1 week on NR 1000 mg twice daily) and were maintained with the 1000 mg twice daily dose through Week 12 (FIG. 19D).

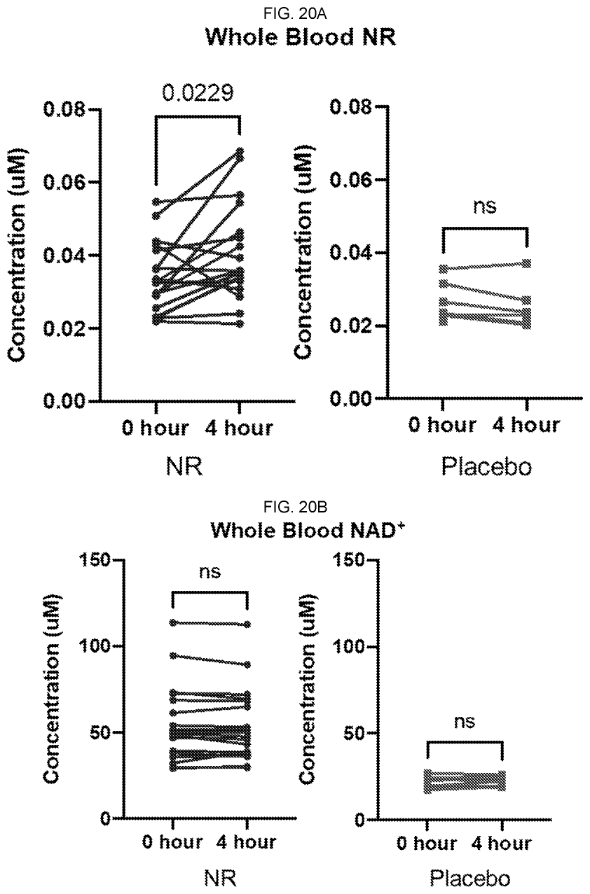

At the Week 4 visit, blood NR and NAD+ levels were obtained just prior to a 1000 mg NR dose, and then repeated at 4 h post-NR dose. While NR levels tended to increase by 4 h following the 1000 mg NR dose (FIG. 20A), NAD+ levels at 4 h were unchanged from their pre-NR dose values (FIG. 20B), similar to the previously-reported findings in healthy volunteers (Airhart S E, et al., PLoS One. 2017, 12:e0186459) This latter observation suggests that NAD+ level increases obtained with NR are sustained across the 12 h dosing interval.

However, as has also been reported previously (Airhart S E, et al., PLoS One. 2017, 12:e0186459), while intra-individual increases in NAD+ levels in response to NR are fairly stable, inter-individual increases in NAD+ levels in response to NR can vary substantially. Mechanisms responsible for this wide, inter-individual variation in NAD+ response to NR treatment are unknown.

Exploratory Endpoints. A power calculation was performed prior to Study initiation, using the reported standard deviation (SD) for change in LVEF by 2D Echo in a study of 50 patients with LV dysfunction due to previous myocardial infarction (Jenkins C, et al., Am J Cardiol. 2007, 99:300-6). This power calculation suggested that a study with 80% power to detect a between-group LVEF difference of 3% would require a total sample size of 172 participants. Thus, the present study was not powered to detect differences in cardiac function endpoints.

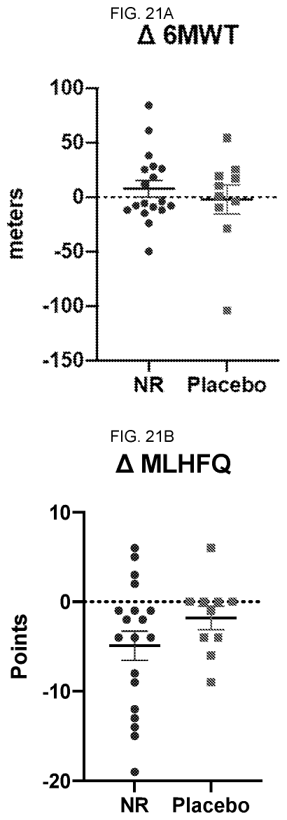

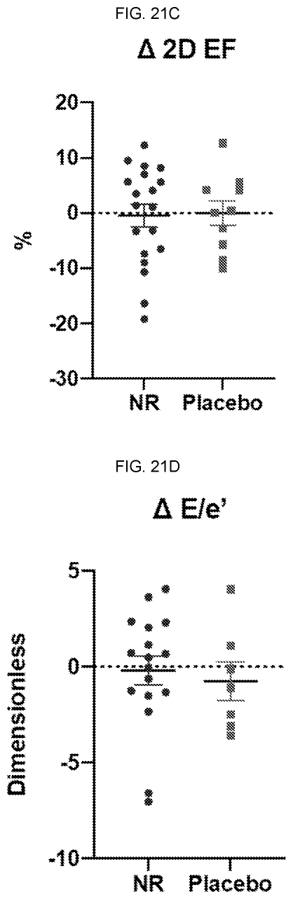

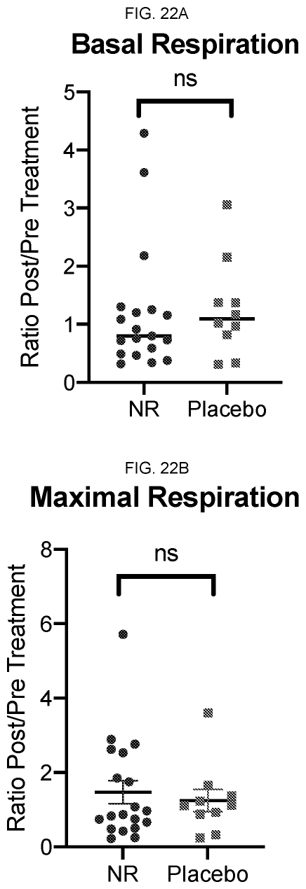

Nonetheless, to investigate a potential range of NR effects on pre-specified surrogate endpoints, serial measurements were obtained for: a) functional capacity, assessed by 6-minute walk test distance, b) quality of life, assessed with the Minnesota Living with Heart Failure Questionnaire (MLHFQ) score, c) left ventricular systolic function, assessed by 2-dimensional left ventricular ejection fraction (2D-LVEF) and d) left ventricular diastolic function (E/e′). No on-trial differences between groups in these exploratory endpoints were detected (FIGS. 21A-21D).

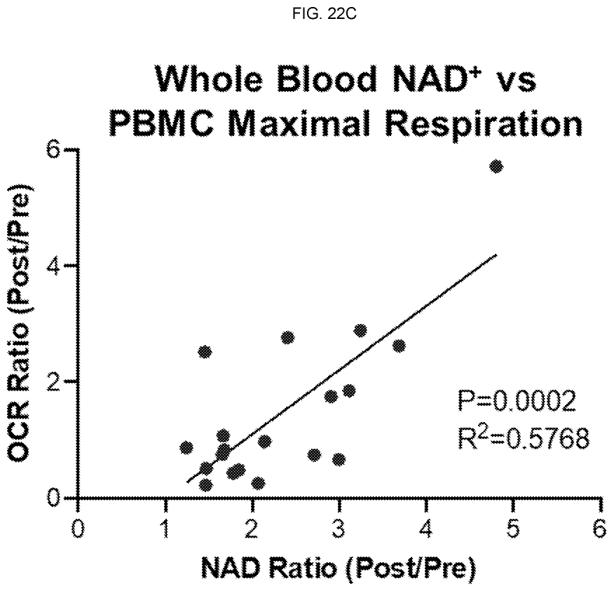

Mitochondrial Respiration in Peripheral Blood Mononuclear Cell (PBMC). Baseline PBMC mitochondrial respiration rates were not changed at Week 12 as compared to Week 0 in either the NR or placebo groups (FIG. 22A). Though the NR group appeared to have a marginal increase by Week 12 vs. Week 0 in maximal PBMC respiration, this difference did not reach statistical significance (FIG. 22B).

In light of the wide, observed variabilities in on-trial NAD+ responses, the relationship between change in NAD+ levels (ratios of Week 12/Week 0 values) and change in maximal mitochondrial respiration (ratios of Week12/Week 0 values) was investigated. In the Placebo group, changes in NAD+vs. maximal mitochondrial respiration were not correlated (R²=0.09358, P=0.39). However, changes in post-/pre-ratios for NAD+ levels and PBMC maximal respiration were highly correlated in the NR group (R²=0.5768, P=0.0002, FIG. 22C).

Discussion. This Study of 30 participants with clinically-stable (ACC/AHA Stage C) HFrEF found that NR has an excellent safety profile and is well-tolerated at a dose up to 1000 mg twice daily over a total treatment duration of 12 weeks. Specifically, there were no significant differences in on-trial rates of either adverse events or serious adverse events or in treatment-emergent laboratory abnormalities. Importantly, NR treatment doubled whole blood NAD+ levels in these clinically-stable patients with HF, a finding similar to those of the previously-reported studies in both healthy volunteers (Airhart S E, et al., PLoS One. 2017, 12:e0186459) and patients with end-stage heart failure (Zhou B, et al., J Clin Invest. 2020, 130:6054-6063 (Experimental Example 1)). The study was underpowered to detect changes in surrogate clinical outcomes. However, a strong correlation was demonstrated between greater increases in on-trial NAD+ levels and improved PBMC maximal mitochondrial respiration, demonstrating a positive, biological effect of orally administered NR in patients with HFrEF.

A feature of failing myocardium is a relative decrease in the ratio of oxidized (NAD+) vs. reduced (NADH) levels of nicotinamide adenine dinucleotide (Karamanlidis G, et al., Cell Metab. 2013, 18:239-50; Lee C F, et al., Circulation. 2016, 134:883-94; Diguet N, et al., Circulation. 2018, 137:2256-2273). In addition to this NAD+/NADH redox imbalance, NAD+ is a co-substrate for multiple enzymes, including sirtuin deacetylases, and plays critical roles in redox balance, cell death, inflammation and post-translational protein modifications important to cellular metabolic processes and energy transduction (Zhou B and R Tian, J Clin Invest. 2018, 128:3716-3726). Recent studies have found that administration of NAD+(Pillai V B, et al., J Biol Chem. 2010, 285:3133-44), the NAD+ precursor, nicotinamide mononucleotide (NMN) (Lee C F, et al., Circulation. 2016, 134:883-94; Martin A S, et al., JCI Insight. 2017, 2) or nicotinamide riboside (NR), an NAD+ salvage pathway precursor (Diguet N, et al., Circulation. 2018, 137:2256-2273) slow disease development in murine models of HF with reduced ejection fraction (HFrEF).

Two recent reports in preclinical models have suggested that targeting NAD+ may also have benefit in heart failure with preserved ejection fraction (HFpEF) (Tong D, et al., Circ Res. 2021, 128(11): 1629-1641; Abdellatif M, et al., Sci Transl Med. 2021, 13(580)). In one, NAD+ levels, were increased either by NR administration of NR or of an activator of nicotinamide phosphoribosyl transferase (NAMPT), which is a key enzyme in the NR “salvage” pathway (Tong D, et al., Circ Res. 2021, 128(11): 1629-1641), In the other study, beneficial effects were seen in another murine HFpEF model using another NAD+ precursor, nicotinamide (NAM) (Abdellatif M, et al., Sci Transl Med. 2021, 13(580)).

Concordant with the observation in this Study suggesting a beneficial effect of NR in improving PBMC mitochondrial function, a recent, small, non-randomized study of participants with Stage D HFrEF also reported an association of NR at the dose employed in this Trial, with improved PBMC mitochondrial respiratory function (Zhou B, et al., J Clin Invest. 2020, 130:6054-6063 (Experimental Example 1)). Interestingly, elevations in circulating cytokine levels are known to correlate with both heart failure severity (Torre-Amione G, et al., J Am Coll Cardiol. 1996, 27:1201-6; Seta Y, et al., J Card Fail. 1996, 2:243-9) and mortality (Mann D L. Circ Res. 2002, 91:988-98; Deswal A, et al., Circulation. 2001, 103:2055-9) and it has recently been reported that NR appeared to decrease PBMC expression of multiple proinflammatory cytokines (Zhou B, et al., J Clin Invest. 2020, 130:6054-6063 (Experimental Example 1)).

Summary. This study finds that, in patients with clinically-stable HFrEF, NR is well-tolerated, appears to have a favorable safety profile, and doubles whole blood NAD+ levels. While the study was underpowered for surrogate endpoints, a post-hoc analysis found that, among those randomized to NR, the relative increase in blood NAD+ levels correlated strongly with degree of improvement in mitochondrial respiratory function, a finding consistent with the previous report in a small, non-randomized cohort of patients with end-stage (ACC/AHA Stage D) HFrEF (Zhou B, et al., J Clin Invest. 2020, 130:6054-6063 (Experimental Example 1)). Therefore, larger studies of patients with HFrEF (and HFpEF) that are powered to assess the effect of NR on clinically-relevant, surrogate endpoints, as well as inflammation, are warranted.

(vii) Closing Paragraphs. Unless otherwise indicated, the practice of the present disclosure can employ conventional techniques of chemistry, organic chemistry, biochemistry, analytical chemistry, and physical chemistry. These methods are described in the following publications. See, e.g., Harcourt, et al., Holt McDougal Modern Chemistry: Student Edition (2018); J. Karty, Organic Chemistry Principles and Mechanisms (2014); Nelson, et al., Lehninger Principles of Biochemistry 5th edition (2008); Skoog, et al., Fundamentals of Analytical Chemistry (8th Edition); Atkins, et al., Atkins' Physical Chemistry (11th Edition).

Each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; 19% of the stated value; 18% of the stated value; 17% of the stated value; ±16% of the stated value; 15% of the stated value; 14% of the stated value; 13% of the stated value; ±12% of the stated value; 11% of the stated value; ±10% of the stated value; 9% of the stated value; 8% of the stated value; 7% of the stated value; 6% of the stated value; 5% of the stated value; 4% of the stated value; ±3% of the stated value; 2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when the application of the meaning renders any construction meaningless or essentially meaningless. In cases where the construction of the term would render it meaningless or essentially meaningless, the definition should be taken from Webster's Dictionary, 3rd Edition, or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method comprising:
identifying a human Stage D heart failure patient;
orally administering to the human Stage D heart failure patient 2000 mg/day of nicotinamide riboside (NR), wherein the 2000 mg/day is split into two 1000 mg/day doses;
obtaining peripheral blood mononuclear cells (PBMCs) from the human Stage D heart failure patient;
measuring the oxygen consumption rate (OCR) of the obtained PBMCs; and
measuring the expression level of interleukin (IL)6, IL1β, and IL18 by the obtained PBMCs.

2. The method of claim 1, wherein the orally administering is for 9 consecutive days.

3. The method of claim 1, wherein the identified human Stage D heart failure patient is hospitalized.

4. A method comprising:
identifying a human subject with a preexisting inflammation marker profile; and
orally administering a high dose regimen of a nicotinamide adenine dinucleotide (NAD) precursor to the human subject,
wherein the high dose regimen of the NAD precursor reduces inflammation markers in the human subject's peripheral blood mononuclear cells (PBMCs).

5. The method of claim 4, wherein the preexisting inflammation marker profile is based on an assessment of the human subject's PBMCs expression level of an inflammatory marker.

6. The method of claim 5, wherein the inflammatory marker is NLRP3.

7. The method of claim 6, wherein the PBMC's NLRP3 expression level is higher than a baseline level.

8. The method of claim 5, wherein the inflammatory marker is IL6.

9. The method of claim 8, wherein the PBMC's IL6 expression level is higher than 6 pg/mL.

10. The method of claim 5, wherein the inflammatory marker is IL1β.

11. The method of claim 10, wherein the PBMC's IL1β expression level is higher than 1.0 pg/mL.

12. The method of claim 5, wherein the inflammatory marker is IL18.

13. The method of claim 12, wherein the PBMC's IL18 expression level is higher than 492 pg/mL.

14. The method of claim 5, wherein the inflammatory marker is TNFα.

15. The method of claim 14, wherein the PBMC's TNFα expression level is higher than 1 pg/mL.

16. The method of claim 5, wherein the inflammatory marker is NLRP3, IL6, IL1β, IL18, and TNFα.

17. The method of claim 16, wherein the PBMC's NLRP3 expression level is higher than a baseline level, the IL6 expression level is higher than 6 pg/mL, the PBMC's IL1β expression level is higher than 1.0 pg/mL, the PBMC's IL18 expression level is higher than 492 pg/mL, and the PBMC's TNFα expression level is higher than 1 pg/mL.

18. The method of claim 4, wherein the reduced inflammation marker in the human subject's PBMCs is an increase in the PBMC's oxygen consumption rate as compared to the PBMC's oxygen consumption rate before administering the high dose regimen.

19. The method of claim 4, wherein the reduced inflammation marker in the human subject's PBMCs is a decrease in the PBMC's NLRP3 expression level as compared to the PBMC's NLRP3 expression level before administering the high dose regimen.

20. The method of claim 4, wherein the reduced inflammation marker in the human subject's PBMCs is a decrease in the PBMC's IL6 expression level as compared to the PBMC's IL6 expression level before administering the high dose regimen.

21. The method of claim 4, wherein the reduced inflammation marker in the human subject's PBMCs is a decrease in the PBMC's IL1β expression level as compared to the PBMC's IL1β expression level before administering the high dose regimen.

22. The method of claim 4, wherein the reduced inflammation marker in the human subject's PBMCs is a decrease in the PBMC's IL18 expression level as compared to the PBMC's IL18 expression level before administering the high dose regimen.

23. The method of claim 4, wherein the reduced inflammation marker in the human subject's PBMCs is a decrease in the PBMC's TNFα expression level as compared to the PBMC's TNFα expression level before administering the high dose regimen.

24. The method of claim 4, wherein the reduced inflammation marker in the human subject's PBMCs is an increase in the PBMC's oxygen consumption rate, and a decrease in the PBMC's expression of NLRP3, IL6, IL1β, IL18, and TNFα as compared to the PBMC's oxygen consumption rate, and NLRP3, IL6, IL1β, IL18, and TNFα expression level before administering the high dose regimen.

25. The method of claim 4, wherein the high dose regimen comprises a dose of the NAD precursor of at least 1000 mg/day.

26. The method of claim 4, wherein the high dose regimen comprises a dose of the NAD precursor of at least 2000 mg/day.

27. The method of claim 26, wherein the high dose regimen comprises the 2000 mg/day dose for at least 5 days, for at least 9 days, for at least 12 days, or for at least 12 weeks.

28. The method of claim 4, wherein the NAD precursor is nicotinamide riboside (NR).

29. The method of claim 4, wherein the NAD precursor is an NR analogue comprising NRH triacetate, NRH triproprionate, NRH tributyrate, NRH triisobutyrate, NR+ tripentanoate, NR+ trihexanoate, NRH triethylcarbonate, NRH tribenzoate, NR+ monohexanoate, NRH monodecanoate, NRH monotetradecanoate, Nic mononucleotide (NMN), NR+ monooleate, NR+ monohexanoate, NR+ monononanoate, NR+ monododecanoate, NR+ monopentanoate, or NR+ monoundecanoate.

30. The method of claim 4, wherein the NAD precursor is NR chloride or a crystalline form of NR chloride.

31. The method of claim 30, wherein the crystalline form of NR chloride comprises
3-carbamoyl-1-((2R,3R,4S,5R)-3,4-dihydroxy-5(hydroxymethy)tetrahydrofuran-2-yl)pyridin-1-ium(β-D-NR) chloride crystal,
3-carbamoyl-1-((2R,3R,4S,5R)-3,4-dihydroxy-5(hydroxymethy)tetrahydrofuran-2-yl)pyridin-1-ium(β-D-NR chloride methanolate crystal, or
3-carbamoyl-1-((2S,3R,4S,5R)-3,4-dihydroxy-5(hydroxymethyl)tetra hydrofuran-2-yl)pyridin-1-ium chloride.

32. The method of claim 4, wherein the NAD precursor is an NR analogue comprising O-ethyl NR (OENR), tri-O-acetyl O′-ethyl NR (TAENR), N-dimethyl NR (DMNR), or N-allyl NR (ANR).

33. The method of claim 4, wherein the human subject with the preexisting inflammation marker profile has a heart condition, an autoimmune disease, cancer, diabetes, a chronic respiratory disease, stroke, obesity, or allergies.

34. The method of claim 33, wherein the heart condition is Stage A, B, C, or D heart failure.

35. The method of claim 34, wherein the heart condition is Stage D heart failure.

36. The method of claim 33, wherein the autoimmune disease is lupus or rheumatoid arthritis.

37. A composition comprising at least 1000 mg of a NAD precursor.

38. The composition of claim 37, comprising 2000 mg of a NAD precursor.

39. The composition of claim 37, wherein the NAD precursor is NR.

40. The composition of claim 37, wherein the NAD precursor is an NR analogue comprising NRH triacetate, NRH triproprionate, NRH tributyrate, NRH triisobutyrate, NR+ tripentanoate, NR+ trihexanoate, NRH triethylcarbonate, NRH tribenzoate, NR+ monohexanoate, NRH monodecanoate, NRH monotetradecanoate, Nic mononucleotide (NMN), NR+ monooleate, NR+ monohexanoate, NR+ monononanoate, NR+ monododecanoate, NR+ monopentanoate, or NR+ monoundecanoate.

41. The composition of claim 37, wherein the NAD precursor is NR chloride or a crystalline form of NR chloride.

42. The composition of claim 37, wherein the crystalline form of NR chloride comprises
3-carbamoyl-1-((2R,3R,4S,5R)-3,4-dihydroxy-5(hydroxymethy)tetrahydrofuran-2-yl)pyridin-1-ium(β-D-NR) chloride crystal,
3-carbamoyl-1-((2R,3R,4S,5R)-3,4-dihydroxy-5(hydroxymethy)tetrahydrofuran-2-yl)pyridin-1-ium(β-D-NR chloride methanolate crystal, or
3-carbamoyl-1-((2S,3R,4S,5R)-3,4-dihydroxy-5(hydroxymethyl)tetra hydrofuran-2-yl)pyridin-1-ium chloride.

43. The composition of claim 37, wherein the NAD precursor is an NR analogue comprising O-ethyl NR (OENR), tri-O-acetyl O′-ethyl NR (TAENR), N-dimethyl NR (DMNR), or N-allyl NR (ANR).
